(19) United States
(12) Patent Application Publication
(10) Pub. No.: US 2026/0216373 A1
(43) Pub. Date: Jul. 30, 2026

(54) Gene Therapy Constructs for the Treatment of Pompe Disease

(71) Applicant: Amicus Therapeutics, Inc., Philadelphia, PA (US)

(72) Inventor: Hung V. Do, New Hope, PA (US)

(73) Assignee: Amicus Therapeutics, Inc., Philadelphia, PA (US)

(21) Appl. No.: 19/147,498

(22) PCT Filed: Jan. 12, 2024
(86) PCT No.: PCT/US2024/011449

§ 371 Date: Jul. 11, 2025

Related U.S. Application Data
(60) Provisional application No. 63/438,960, filed on Jan. 13, 2023.

(51) Int. Cl. A61K 48/00; A61K 9/00; A61K 38/00; A61P 3/00; C07K 14/65; C12N 9/24; C12N 15/86

(52) U.S. Cl. CPC A61K 48/005; A61K 9/0019; A61K 48/0075; A61K 48/0083; A61P 3/00; C07K 14/65; C12N 9/2402; C12N 15/86; A61K 38/00; C07K 2319/02; C12N 2750/14143; C12Y 302/0102

(57) ABSTRACT

The present disclosure relates to recombinant adeno-associated virus (rAAV) delivery of an acid alpha-glucosidase (GAA) polynucleotide. The disclosure provides rAAV vectors and methods of using the rAAV vectors for GAA gene therapy for Pompe disease.

### FIELD

The present invention generally relates to the treatment of lysosomal storage disorders, particularly gene therapy for the treatment of Pompe disease.

### BACKGROUND

Genetic disorders arise via heritable or de novo mutations occurring in gene coding regions of the genome. For example, Pompe disease, a debilitating neuromuscular illness that affects infants, children, and adults with different degrees of severity, is caused by a deficiency of lysosomal glycogen-degrading enzyme acid alpha glucosidase (GAA).

In some cases, such genetic disorders are treated by administration of a protein encoded by the gene mutated in the individual having the genetic disorder. Such treatment has challenges however, as administration of the protein does not always result in the protein reaching the organs, cells, or organelle where it is needed. Furthermore, this treatment also often requires weekly or biweekly infusions, which are not needed with gene therapy, where a single treatment can offer lasting relief.

Therefore, gene therapy has the potential to offer improved results over currently available treatments for genetic disorders.

### SUMMARY

Provided herein are gene therapy vectors such as recombinant adeno-associated virus (rAAV) encoding an acid alpha glucosidase (GAA) polypeptide for the treatment of Pompe disease.

One aspect of the present invention relates to a gene therapy vector comprising a nucleic acid construct encoding a polypeptide comprising: a therapeutic protein comprising acid alpha-glucosidase (GAA); a CIMPR-binding peptide comprising a variant IGF2 (vIGF2) peptide; and a signal peptide comprising a binding immunoglobulin protein (BiP) signal peptide. Exemplary vIGF2 peptides, BiP signal peptides, and linkers are described in PCT/US2012/039705 and U.S. Pat. No. 10,874,750, which are hereby incorporated by reference in their entireties.

In one or more embodiments, the gene therapy vector further comprises an adeno-associated viral (AAV) vector. Exemplary AAV vectors are described in PCT/US2020/030484 and PCT/US2020/030493, which are hereby incorporated by reference in their entirety. In some embodiments, the AAV vector is an AAV9 vector.

In some embodiments, the gene therapy vector further comprises a promoter. An exemplary promoter includes cytomegalovirus (CMV) early enhancer/chicken R actin (CAG) promoter.

In some embodiments, the methods described herein deliver an enzyme fused to a vIGF2 or fused to a signal peptide to the patient in order to deliver the enzyme to the cell where it is needed. In some embodiments, any of the nucleic acid constructs provided herein further comprise a nucleic acid sequence encoding a peptide that selectively binds to the CIMPR with high affinity, wherein the therapeutic protein and the peptide that selectively binds to the CIMPR are expressed as a fusion protein. In some embodiments, the nucleic acid construct further comprises a sequence encoding a linker peptide between the nucleic acid encoding the peptide that selectively binds to the CIMPR nucleotide sequence and the nucleic acid sequence encoding the therapeutic protein. In some embodiments, the sequence of the linker peptide may overlap with the sequence of the therapeutic peptide or the sequence of the peptide that selectively binds to the CIMPR, or both. In certain aspects, there are provided gene therapy vectors, such as gene therapy vectors comprising a nucleic acid sequence encoding a peptide that binds to the CIMPR with high affinity. In some embodiments, the peptide that binds to CIMPR with high affinity is a variant IGF2 peptide (vIGF2).

In some embodiments, the vIGF2 peptide has decreased or no affinity for the insulin receptor and IGF1R as compared to native IGF2 peptide. In some embodiments, the vIGF2 peptide is capable of facilitating uptake of the therapeutic protein into a cell. In some embodiments, the vIGF2 peptide is capable of facilitating uptake of the therapeutic protein into a lysosome. In one aspect, the vector is capable of expressing the vIGF2-therapeutic fusion protein construct in mammalian cells.

In some embodiments, the nucleic acid encoding the therapeutic fusion protein, such as a vIGF2 fusion, optionally has an internal ribosomal entry sequence, and is cloned into a number of types of vectors. For example, in some embodiments, the nucleic acid is cloned into a vector including, but not limited to a plasmid, a phagemid, a phage derivative, an animal virus, and a cosmid. Vectors of particular interest include expression vectors, replication vectors, probe generation vectors, and sequencing vectors. Another aspect of the present invention relates to a method of treating Pompe disease in a subject, the method comprising administering a therapeutically effective amount of a gene therapy vector as described herein.

In some embodiments, the gene therapy vector is administered systemically. In some embodiments, wherein the gene therapy vector is administered via a route selected from the group consisting of intrathecal, intracisternal, lumbar puncture, intracranial, intracerebroventricular, intraparenchymal, intravenous and combinations thereof.

In some embodiments, the therapeutically effective amount of the gene therapy vector is in a range of from about 1×10¹² to about 1×10¹⁵ vg. In some embodiments, the therapeutically effective amount of the gene therapy vector is in a range of from about 5×10¹² to about 1×10¹⁴ vg. In some embodiments, the therapeutically effective amount of the composition is about 2.5×10¹³ vg or about 5×10¹³ vg. In some embodiments, the therapeutically effective amount of the composition is about 2.5×10¹³ vg. In some embodiments, the therapeutically effective amount of the composition is about 5×10¹³ vg.

In some embodiments, gene therapy vector is not administered in combination with a non-ionic, low-osmolar contrast agent. In some embodiments, gene therapy vector is administered in combination with a non-ionic, low-osmolar contrast agent. In some embodiments, the non-ionic, low-osmolar contrast agent is selected from the group consisting of iobitridol, iohexol, iomeprol, iopamidol, iopentol, iopromide, ioversol, ioxilan, and combinations thereof.

In some embodiments, the method reduces glycogen storage in patient cells. In some embodiments, the method reduces glycogen storage in one or more of skeletal and cardiac muscles, the diaphragm, and the central nervous system (CNS).

### DETAILED DESCRIPTION

The present disclosure provides methods and products for treating Pompe disease. The methods involve delivery of a GAA+vIGF2 polynucleotide to a subject using a gene delivery vector such as AAV.

Adeno-associated virus (AAV) is a replication-deficient parvovirus, the single-stranded DNA genome of which is about 4.7 kb in length including 145 nucleotide inverted terminal repeat (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest (e.g., a GAA-encoding polynucleotide) flanked on the 5′ and 3′ ends by inverted terminal repeat (ITR). In some embodiments, the rAAV genome comprises a “gene cassette.” The rAAV genome can be a self-complementary (sc) genome, which is referred to herein as “scAAV genome.” Alternatively, the rAAV genome can be a single-stranded (ss) genome, which is referred to herein as “ssAAV genome.”

The term “scAAV” refers to an rAAV virus or rAAV viral particle comprising a self-complementary genome. The term “ssAAV” refers to an rAAV virus or rAAV viral particle comprising a single-stranded genome.

rAAV genomes provided herein may comprise a polynucleotide encoding a GAA polypeptide. GAA polypeptides can comprise the amino acid sequence set out in SEQ ID NO: 1, 2, 3 or 4, or a polypeptide with an amino acid sequence that is at least: 90%, 91%, 92%, 93%, 94%, 95%, 96%, 97%, 98%, or 99% identical to the amino acid sequence set forth in SEQ ID NO: 1, 2, 3 or 4, and which encodes a polypeptide with GAA activity (e.g. breaking down glycogen).

TABLE 1
Nucleotide Sequences and Protein Sequences

| SEQ ID | |
|---|---|
| NO | Sequences |
| 1 | MGVRHPPCSHRLLAVCALVSLATAALLGHILLHDFLLVPRELSGSSPVLEETHPAHQQGA |
| | SRPGPRDAQAHPGRPRAVPTQCDVPPNSREDCAPDKAITQEQCEARGCCYIPAKQGLQGA |
| | QMGQPWCFFPPSYPSYKLENLSSSEMGYTATLTRTTPTFFPKDILTLRLDVMMETENRLH |
| | FTIKDPANRRYEVPLETPRVHSRAPSPLYSVEFSEEPFGVIVHRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTENKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPI |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 2 | MGVRHPPCSHRLLAVCALVSLATAALLGHILLHDFLLVPRELSGSSPVLEETHPAHQQGA |
| | SRPGPRDAQAHPGRPRAVPTQCDVPPNSRFDCAPDKAITQEQCEARGCCYIPAKQGLOGA |
| | QMGQPWCFFPPSYPSYKLENLSSSEMGYTAILTRTTPTFFPKDILTLRLDVMMETENRLH |
| | FTIKDPANRRYEVPLETPHVHSRAPSPLYSVEFSEEPFGVIVRRQLDGRVLLNTTVAPLF |
| | FADQFLQLSTSLPSQYITGLAEHLSPLMLSTSWTRITLWNRDLAPTPGANLYGSHPFYLA |
| | LEDGGSAHGVFLLNSNAMDVVLQPSPALSWRSTGGILDVYIFLGPEPKSVVQQYLDVVGY |
| | PFMPPYWGLGFHLCRWGYSSTAITRQVVENMTRAHFPLDVQWNDLDYMDSRRDFTENKDG |
| | FRDFPAMVQELHQGGRRYMMIVDPAISSSGPAGSYRPYDEGLRRGVFITNETGQPLIGKV |
| | WPGSTAFPDFTNPTALAWWEDMVAEFHDQVPFDGMWIDMNEPSNFIRGSEDGCPNNELEN |
| | PPYVPGVVGGTLQAATICASSHQFLSTHYNLHNLYGLTEAIASHRALVKARGTRPFVISR |
| | STFAGHGRYAGHWTGDVWSSWEQLASSVPEILQFNLLGVPLVGADVCGFLGNTSEELCVR |
| | WTQLGAFYPFMRNHNSLLSLPQEPYSFSEPAQQAMRKALTLRYALLPHLYTLFHQAHVAG |
| | ETVARPLFLEFPKDSSTWTVDHQLLWGEALLITPVLQAGKAEVTGYFPLGTWYDLQTVPV |
| | EALGSLPPPPAAPREPAIHSEGQWVTLPAPLDTINVHLRAGYIIPLQGPGLTTTESRQQP |
| | MALAVALTKGGEARGELFWDDGESLEVLERGAYTQVIFLARNNTIVNELVRVTSEGAGLQ |
| | LQKVTVLGVATAPQQVLSNGVPVSNFTYSPDTKVLDICVSLLMGEQFLVSWC |
| 3 | QQGASRPGPRDAQAHPGRPRAVPTQCDVPPNSREDCAPDKAITQEQCEARGCCYIPAKQG |
| | LQGAQMGQPWCFFPPSYPSYKLENLSSSEMGYTAILTRTTPTFFPKDILTLRLDVMMETE |
| | NRLHFTIKDPANRRYEVPLETPRVHSRAPSPLYSVEFSEEPFGVIVHRQLDGRVLLNTTV |
| | APLFFADQFLQLSTSLPSQYITGLAEHLSPLMLSTSWTRITLWNRDLAPTPGANLYGSHP |
| | FYLALEDGGSAHGVFLLNSNAMDVVLQPSPALSWRSTGGILDVYIFLGPEPKSVVQQYLD |
| | VVGYPFMPPYWGLGFHLCRWGYSSTAITRQVVENMTRAHFPLDVQWNDLDYMDSRRDFTF |
| | NKDGFRDFPAMVQELHQGGRRYMMIVDPAISSSGPAGSYRPYDEGLRRGVFITNETGQPL |
| | IGKVWPGSTAFPDFTNPTALAWWEDMVAEFHDQVPFDGMWIDMNEPSNFIRGSEDGCPNN |
| | ELENPPYVPGVVGGTLQAATICASSHQFLSTHYNLHNLYGLTEAIASHRALVKARGTRPF |
| | VISRSTFAGHGRYAGHWTGDVWSSWEQLASSVPEILQFNLLGVPLVGADVCGFLGNTSEE |
| | LCVRWTQLGAFYPFMRNHNSLLSLPQEPYSFSEPAQQAMRKALTLRYALLPHLYTLFHQA |
| | HVAGETVARPLFLEFPKDSSTWTVDHQLLWGEALLITPVLQAGKAEVTGYFPLGTWYDLQ |
| | TVPIEALGSLPPPPAAPREPAIHSEGQWVTLPAPLDTINVHLRAGYIIPLQGPGLITTES |
| | RQQPMALAVALTKGGEARGELFWDDGESLEVLERGAYTQVIFLARNNTIVNELVRVTSEG |
| | AGLQLQKVTVLGVATAPQQVLSNGVPVSNFTYSPDTKVLDICVSLLMGEQFLVSWC |
| 4 | QQGASRPGPRDAQAHPGRPRAVPTQCDVPPNSREDCAPDKAITQEQCEARGCCYIPAKQGLQG |
| | AQMGQPWCFFPPSYPSYKLENLSSSEMGYTATLTRTTPTFFPKDILTLRLDVMMETENRLHFT |
| | IKDPANRRYEVPLETPHVHSRAPSPLYSVEFSEEPFGVIVRRQLDGRVLLNTTVAPLFFADQF |
| | LQLSTSLPSQYITGLAEHLSPLMLSTSWTRITLWNRDLAPTPGANLYGSHPFYLALEDGGSAH |
| | GVFLLNSNAMDVVLQPSPALSWRSTGGILDVYIFLGPEPKSVVQQYLDVVGYPFMPPYWGLGF |
| | HLCRWGYSSTAITRQVVENMTRAHFPLDVQWNDLDYMDSRRDFTENKDGFRDFPAMVQELHQG |
| | GRRYMMIVDPAISSSGPAGSYRPYDEGLRRGVFITNETGQPLIGKVWPGSTAFPDFTNPTALA |
| | WWEDMVAEFHDQVPFDGMWIDMNEPSNFIRGSEDGCPNNELENPPYVPGVVGGILQAATICAS |
| | SHQFLSTHYNLHNLYGLTEAIASHRALVKARGTRPFVISRSTFAGHGRYAGHWTGDVWSSWEQ |
| | LASSVPEILQFNLLGVPLVGADVCGFLGNTSEELCVRWTQLGAFYPFMRNHNSLLSLPQEPYS |
| | FSEPAQQAMRKALTLRYALLPHLYTLFHQAHVAGETVARPLFLEFPKDSSTWTVDHQLLWGEA |
| | LLITPVLQAGKAEVTGYFPLGTWYDLQTVPVEALGSLPPPPAAPREPAIHSEGQWVTLPAPLD |
| | TINVHLRAGYIIPLQGPGLTTTESRQQPMALAVALTKGGEARGELFWDDGESLEVLERGAYTQ |
| | VIFLARNNTIVNELVRVTSEGAGLQLQKVTVLGVATAPQQVLSNGVPVSNFTYSPDTKVLDIC |
| | VSLLMGEQFLVSWC |

The rAAV genomes provided herein, in some embodiments, comprise one or more AAV JTRs flanking the polynucleotide encoding a GAA polypeptide. The GAA polynucleotide is operatively linked to transcriptional control elements (including, but not limited to, promoters, enhancers and/or polyadenylation signal sequences) that are functional in target cells to form a gene cassette. Examples of promoters are the P546 promoter and the chicken β actin (CAG) promoter. Additional promoters are contemplated herein including, but not limited to the simian virus 40 (SV40) early promoter, mouse mammary tumor virus (MMTV), human immunodeficiency virus (HJV) long terminal repeat (LTR) promoter, MoMuLV promoter, an avian leukemia virus promoter, an Epstein-Barr virus immediate early promoter, a Rous sarcoma virus promoter, as well as human gene promoters such as but not limited to, the actin promoter, the myosin promoter, the elongation factor-1a promoter, the hemoglobin promoter, and the creatine kinase promoter. Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a GAA RNA transcript when expressed in mammalian cells. One example of such an intron is the SV40 intron.

“Packaging” refers to a series of intracellular events that result in the assembly and encapsidation of an AAV particle.

The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allows AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV particles, in which an AAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692, which is incorporated by reference herein in its entirety. In various embodiments, AAV capsid proteins may be modified to enhance delivery of the rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. An rAAV with a single-stranded genome is referred to herein as an ssAAV.

An exemplary rAAV vector provided herein is named “AAV9.CAG.BiP.vIGF2.hGAAco(V810I).rBG.KanR.” The rAAV vector AAV9.CAG.BiP.vIGF2.hGAAco(V810I).rBG.KanR comprises the CAG promoter, a polynuloetide encoding a BiP signal peptide, a polynuloetide encoding a vIGF2 peptide and a polynuloetide encoding a human GAA polypeptide.

The rAAV may be purified by methods standard in the art, such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions can comprise a rAAV encoding a GAA polypeptide. Compositions may include two or more rAAV encoding different polypeptides of interest. In some embodiments, the rAAV is scAAV or ssAAV.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20% to about 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 20% to about 40% non-ionic, low-osmolar compound. Another exemplary composition comprises scAAV formulated in and 1×PBS and 0.001% Pluronic F68. In some embodiments, the composition does not comprise a non-ionic, low-osmolar contrast agent. In some embodiments, the composition can be prepared and administered using a therapeutically effective procedure, including by mixing the scAAV9 genome with the contrast agent prior to delivering to the subject. In another embodiment, the scAAV9 genome and the contrasting contrast agent are delivered sequentially.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include from about 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.4×10¹³, about 3.5×10¹³, about 4×10¹³, about 4.5×10¹³, about 5×10¹³, about 6×10¹³, about 1×10¹⁴, about 1.2×10¹⁴, about 2×10¹⁴, about 3×10¹⁴, about 4×10¹⁴, about 5×10¹⁴, about 1×10¹⁵, to about 1×10¹⁶, or more total viral genomes. Dosages of about 1×10¹¹ to about 1×10¹⁵ vg, about 1×10¹² to about 1×10¹⁵ vg, about 1×10¹² to about 1×10¹⁴ vg, about 1×10¹³ to about 1×10¹⁴ vg are also contemplated. One dose exemplified herein 5×10¹² vg (0.5×10¹³ vg). Another dose exemplified herein includes 2.5×10¹³ vg. Another dose exemplified herein includes 5×10¹⁴ vg.

Methods of transducing target cells (including, but not limited to, cells of the nervous system, nerve or glial cells) with rAAV are provided. The cells of the nervous system include neurons, lower motor neurons, microglial cells, oligodendrocytes, astrocytes, Schwann cells or combinations thereof.

The term “transduction” is used to refer to the administration/delivery of the GAA polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a GAA polypeptide by an intrathecal, intracisternal, lumbar puncture, intracranial, intracerebroventricular, intraparenchymal, intravenous and combinations thereof. Intrathecal delivery refers to delivery into the space under the arachnoid membrane of the brain or spinal cord. In some embodiments, intrathecal administration is via intracisternal administration. In some embodiments, intrathecal administration is via intra cisterna magna (ICM) administration. In some embodiments, the intra cisterna magna (ICM) administration is at the craniocervical junction. In some embodiments, the intra cisterna magna (ICM) administration is at the suboccipital region.

Provided herein are peptides that bind CIMPR to comprise a polypeptide which encodes a nucleic acid construct for a gene therapy vector. In some embodiments, the peptide is a vIGF2 peptide. Some vIGF2 peptides maintain high affinity binding to CIMPR while their affinity for IGF1 receptor, insulin receptor, and IGF binding proteins (IGFBP) is decreased or eliminated. Thus, some variant IGF2 peptides are substantially more selective and have reduced safety risks compared to wt IGF2. vIGF2 peptides herein include those having the amino acid sequence of SEQ ID NO: 17. Variant IGF2 peptides further include those with variant amino acids at positions 6, 26, 27, 43, 48, 49, 50, 54, 55, or 65 compared to wt IGF2 (SEQ ID NO: 5). In some embodiments, the vIGF2 peptide has a sequence having one or more substitutions from the group consisting of E6R, F26S, Y27L, V43L, F48T, R49S, S50I, A54R, L55R, and K65R. In some embodiments, the vIGF2 peptide has a sequence having a substitution of E6R. In some embodiments, the vIGF2 peptide has a sequence having a substitution of F26S. In some embodiments, the vIGF2 peptide has a sequence having a substitution of Y27L. In some embodiments, the vIGF2 peptide has a sequence having a substitution of V43L. In some embodiments, the vIGF2 peptide has a sequence having a substitution of F48T. In some embodiments, the vIGF2 peptide has a sequence having a substitution of R49S. In some embodiments, the vIGF2 peptide has a sequence having a substitution of S50I. In some embodiments, the vIGF2 peptide has a sequence having a substitution of A54R. In some embodiments, the vIGF2 peptide has a sequence having a substitution of L55R. In some embodiments, the vIGF2 peptide has a sequence having a substitution of K65R. In some embodiments, the vIGF2 peptide has a sequence having a substitution of E6R, F26S, Y27L, V43L, F48T, R49S, S50I, A54R, and L55R. In some embodiments, the vIGF2 peptide has an N-terminal deletion. In some embodiments, the vIGF2 peptide has an N-terminal deletion of one amino acid. In some embodiments, the vIGF2 peptide has an N-terminal deletion of two amino acids. In some embodiments, the vIGF2 peptide has an N-terminal deletion of three amino acids. In some embodiments, the vIGF2 peptide has an N-terminal deletion of four amino acids. In some embodiments, the vIGF2 peptide has an N-terminal deletion of four amino acids and a substitution of E6R, Y27L, and K65R. In some embodiments, the vIGF2 peptide has an N-terminal deletion of four amino acids and a substitution of E6R and Y27L. In some embodiments, the vIGF2 peptide has an N-terminal deletion of five amino acids. In some embodiments, the vIGF2 peptide has an N-terminal deletion of six amino acids. In some embodiments, the vIGF2 peptide has an N-terminal deletion of seven amino acids. In some embodiments, the vIGF2 peptide has an N-terminal deletion of seven amino acids and a substitution of Y27L and K65R.

In some embodiments, the vIGF2 peptide comprises an N-terminal deletion. In some embodiments, the vIGF2 peptide comprises an N-terminal deletion at position 1 of SEQ ID NO: 5. In some embodiments, the vIGF2 peptide comprises an N-terminal deletion of positions 1-2 of SEQ ID NO: 5. In some embodiments, the vIGF2 peptide comprises an N-terminal deletion of positions 1-3 of SEQ ID NO: 5. In some embodiments, the vIGF2 peptide comprises an N-terminal deletion of positions 1-4 of SEQ ID NO: 5. In some embodiments, the vIGF2 peptide comprises an N-terminal deletion of positions 1-4 of SEQ ID NO: 5 and substitutions of E6R, Y27L, and K65R. In some embodiments, the vIGF2 peptide comprises an N-terminal deletion of positions 1-4 of SEQ ID NO: 5 and substitutions of E6R and Y27L. In some embodiments, the vIGF2 peptide comprises an N-terminal deletion of positions 1-5 of SEQ ID NO: 5. In some embodiments, the vIGF2 peptide comprises an N-terminal deletion at positions 1-6 of SEQ ID NO: 5. In some embodiments, the vIGF2 peptide comprises an N-terminal deletion at positions 1-7 of SEQ ID NO: 5.

In some embodiments, the vIGF2 peptide comprises an amino acid sequence that is at least 90% identical to SEQ ID NO: 5 and having at least one substitution at one or more positions selected from the group consisting of positions 6, 26, 27, 43, 48, 49, 50, 54, 55, and 65 of SEQ ID NO: 5. In some embodiments, the at least one substitution is selected from the group consisting of E6R, F26S, Y27L, V43L, F48T, R49S, S50I, A54R, L55R, and K65R of SEQ ID NO: 5. In some embodiments, the at least one substitution is selected from the group consisting of E6R, F26S, Y27L, V43L, F48T, R49S, S50I, A54R, and L55R of SEQ ID NO: 5. In some embodiments, the vIGF2 peptide comprises at least two substitutions at two or more positions selected from the group consisting of positions 6, 26, 27, 43, 48, 49, 50, 54, and 55 of SEQ ID NO: 5. In some embodiments, the at least two substitutions are selected from the group consisting of E6R, F26S, Y27L, V43L, F48T, R49S, S50I, A54R, and L55R of SEQ ID NO: 5. In some embodiments, the vIGF2 peptide comprises an N-terminal deletion. In some embodiments, the vIGF2 peptide comprises an N-terminal deletion at position 1 of SEQ ID NO: 5. In some embodiments, the vIGF2 peptide comprises an N-terminal deletion at positions 1-6 of SEQ ID NO: 5. In some embodiments, the vIGF2 peptide comprises an N-terminal deletion of positions 1-2 of SEQ ID NO: 5. In some embodiments, the vIGF2 peptide comprises an N-terminal deletion of positions 1-3 of SEQ ID NO: 5. In some embodiments, the vIGF2 peptide comprises an N-terminal deletion of positions 1-4 of SEQ ID NO: 5. In some embodiments, the vIGF2 peptide comprises an N-terminal deletion of positions 1-4 of SEQ ID NO: 5 and a substitution of E6R, Y27L, and K65R. In some embodiments, the vIGF2 peptide comprises an N-terminal deletion of positions 1-4 of SEQ ID NO: 5 and a substitution of E6R and Y27L. In some embodiments, the vIGF2 peptide comprises an N-terminal deletion of positions 1-5 of SEQ ID NO: 5. In some embodiments, the vIGF2 peptide comprises an N-terminal deletion of positions 1-6 of SEQ ID NO: 5. In some embodiments, the vIGF2 peptide comprises an N-terminal deletion at positions 1-7 of SEQ ID NO: 5. In some embodiments, the vIGF2 peptide has increased specificity for the cation-independent M6P receptor (CIMPR) as compared to native IGF2 peptide. In some embodiments, the vIGF2 peptide is capable of facilitating uptake of the therapeutic protein into a lysosome in a cell.

In some embodiments, the vIGF2 peptide comprises an amino acid sequence at least 90% identical to an amino acid sequence selected from the group consisting of SEQ ID NOs: 5-15. In some embodiments, the vIGF2 peptide comprises an amino acid sequence selected from the group consisting of SEQ ID NOs: 6-15. In some embodiments, the vIGF2 nucleotide sequence is 5′ to the nucleic acid sequence encoding a therapeutic protein. In some embodiments, the vIGF2 nucleotide sequence is 3′ to the nucleic acid sequence encoding a therapeutic protein.

In some embodiments, the vIGF2 peptide comprises an amino acid sequence that is at least 90% identical to SEQ ID NO: 5 and having at least one substitution at one or more positions selected from the group consisting of positions 6, 26, 27, 43, 48, 49, 50, 54, 55, and 65 of SEQ ID NO: 5. In some embodiments, the at least one substitution is selected from the group consisting of E6R, F26S, Y27L, V43L, F48T, R49S, S50I, A54R, L55R, and K65R of SEQ ID NO: 5. In some embodiments, the vIGF2 peptide comprises at least two substitutions at two or more positions selected from the group consisting of positions 6, 26, 27, 43, 48, 49, 50, 54, 55, 65 of SEQ ID NO: 5. In some embodiments, the at least two substitutions are selected from the group consisting of E6R, F26S, Y27L, V43L, F48T, R49S, S50I, A54R, L55R, K65R of SEQ ID NO: 5. In some embodiments, the vIGF2 peptide comprises an N-terminal deletion at positions 1-4 of SEQ ID NO: 5. In some embodiments, the vIGF2 peptide comprises the sequence of SEQ ID NO: 17. In some embodiments, wherein the vIGF2 peptide has decreased affinity for insulin receptor and IGF1R as compared to native IGF2 peptide. In some embodiments, the vIGF2 peptide is capable of facilitating uptake of the therapeutic protein into a cell. In some embodiments, the vIGF2 peptide is capable of facilitating uptake of the therapeutic protein into a lysosome. In some embodiments, the therapeutic protein is capable of replacing a defective or deficient protein associated with a genetic disorder in a subject having Pompe Disease.

TABLE 2
IGF 2Amino Acid Sequences (variant residues are underlined)

| SEQ | | |
|---|---|---|
| ID | | |
| NO | Peptide | Sequences |
| 5 | Wild- | AYRPSETLCGGELVDTLQFVCGDRGFYFSRPASRVSRRSRGIVEECCFRSCDLALLETY |
| | type | CATPAKSE |
| 6 | F26S | AYRPSETLCGGELVDTLQFVCGDRGSYFSRPASRVSRRSRGIVEECCFRSCDLALLETY |
| | | CATPAKSE |
| 7 | Y27L | AYRPSETLCGGELVDTLQFVCGDRGFLFSRPASRVSRRSRGIVEECCFRSCDLALLETY |
| | | CATPAKSE |
| 8 | V43L | AYRPSETLCGGELVDTLQFVCGDRGFYFSRPASRVSRRSRGILEECCFRSCDLALLETY |
| | | CATPAKSE |
| 9 | F48T | AYRPSETLCGGELVDTLQFVCGDRGFYFSRPASRVSRRSRGIVEECCTRSCDLALLETY |
| | | CATPAKSE |
| 10 | R49S | AYRPSETLCGGELVDTLQFVCGDRGFYFSRPASRVSRRSRGIVEECCFSSCDLALLETY |
| | | CATPAKSE |
| 11 | S50I | AYRPSETLCGGELVDTLQFVCGDRGFYFSRPASRVSRRSRGIVEECCFRICDLALLETY |
| | | CATPAKSE |
| 12 | A54R | AYRPSETLCGGELVDTLQFVCGDRGFYFSRPASRVSRRSRGIVEECCFRSCDLRLLETY |
| | | CATPAKSE |
| 13 | L55R | AYRPSETLCGGELVDTLQFVCGDRGFYFSRPASRVSRRSRGIVEECCFRSCDLARLETY |
| | | CATPAKSE |
| 14 | F26S, | AYRPSETLCGGELVDTLQFVCGDRGSLFSRPASRVSRRSRGILEECCTSICDLRRLETY |
| | Y27L, | CATPAKSE |
| | V43L, | |
| | F48T, | |
| | R49S, | |
| | S50I, | |
| | A54R, | |
| | L55R | |
| 15 | Δ1-6, | TLCGGELVDTLQFVCGDRGFLFSRPASRVSRRSRGIVEECCFRSCDLALLETYCATPAR |
| | Y27L, | SE |
| | K65R | |
| 16 | Δ1-7, | LCGGELVDTLQFVCGDRGFLFSRPASRVSRRSRGIVEECCFRSCDLALLETYCATPARS |
| | Y27L, | E |
| | K65R | |
| 17 | Δ1-4, | SRTLCGGELVDTLQFVCGDRGFLFSRPASRVSRRSRGIVEECCFRSCDLALLETYCATP |
| | E6R, | ARSE |
| | Y27L, | |
| | K65R | |
| 18 | Δ1-4, | SRTLCGGELVDTLQFVCGDRGFLFSRPASRVSRRSRGIVEECCFRSCDLALLETYCATP |
| | E6R, | AKSE |
| | Y27L | |
| 19 | E6R | AYRPSRTLCGGELVDTLQFVCGDRGFYFSRPASRVSRRSRGIVEECCFRSCDLALLETY |
| | | CATPAKSE |

TABLE 3
IGF2 DNA Coding Sequences

| SEQ ID | | |
|---|---|---|
| NO | Peptide | DNA Sequence |
| 25 | Mature | GCTTACCGCCCCAGTGAGACCCTGTGCGGCGG |
| | WT | GGAGCTGGTGGACACCCTCCAGTTCGTCTGTG |
| | IGF2 | GGGACCGCGGCTTCTACTTCAGCAGGCCCGCA |
| | | AGCCGTGTGAGCCGTCGCAGCCGTGGCATCGT |
| | | TGAGGAGTGCTGTTTCCGCAGCTGTGACCTGG |
| | | CCCTCCTGGAGACGTACTGTGCTACCCCCGCC |
| | | AAGTCCGAG |
| 26 | vIGF2 | TCTAGAACACTGTGCGGAGGGGAGCTTGTAGA |
| | Δ1-4, | CACTCTTCAGTTCGTGTGTGGAGATCGCGGGT |
| | E6R, | TCCTCTTCTCTCGCCCCGCTTCCAGAGTTTCA |
| | Y27L, | CGGAGGTCTAGGGGTATAGTAGAGGAGTGTTG |
| | K65R | TTTCAGGTCCTGTGACTTGGCGCTCCTCGAGA |
| | | CCTATTGCGCGACGCCAGCCAGGTCCGAA |

In some embodiments, the nucleic acid construct further comprises a nucleic acid sequence encoding a signal peptide capable of increasing secretion of the therapeutic protein as compared to the therapeutic protein without the signal peptide. In some embodiments, the signal peptide is a binding immunoglobulin protein (BiP) signal peptide. In some embodiments, the BiP signal peptide comprises an amino acid sequence at least 90% identical to an amino acid sequence selected from the group consisting of SEQ ID Nos: 20-24. In some embodiments, the signal peptide differs from a sequence selected from the group consisting of SEQ ID Nos: 20-24 by 5 or fewer, 4 or fewer, 3 or fewer, 2 or fewer, or 1 amino acid. In some embodiments, the BiP signal peptide comprises an amino acid sequence selected from the group consisting of SEQ ID Nos: 20-24.

TABLE 4
BiP Signal Peptide Sequences

| SEQ ID NO | Signal | Peptide | Amino Acid Sequence |
|---|---|---|---|
| 20 | Native BiP | Human | MKLSLVAAMLLLLSAARA |
| 21 | Modified | BiP-1 | MKLSLVAAMLLLLSLVAAMLLLLSAARA |
| 22 | Modified | BiP-2 | MKLSLVAAMLLLLWVALLLLSAARA |
| 23 | Modified | BiP-3 | MKLSLVAAMLLLLSLVALLLLSAARA |
| 24 | Modified | BiP-4 | MKLSLVAAMLLLLALVALLLLSAARA |

The methods provided herein comprise the step of administering an effective dose, or effective multiple doses, of a composition comprising a rAAV provided herein to a subject (e.g., an animal including, but not limited to, a human patient) in need thereof. If the dose is administered prior to development of Pompe disease, the administration is prophylactic. If the dose is administered after the development of Pompe disease, the administration is therapeutic. An effective dose is a dose that alleviates (eliminates or reduces) at least one symptom associated with the disease, that slows or prevents progression of the disease, that diminishes the extent of disease, that results in remission (partial or total) of disease, and/or that prolongs survival. In comparison to the subject before treatment or in comparison to an untreated subject, methods provided herein result in stabilization, reduced progression, or improvement in one or more of the scales that are used to evaluate progression and/or improvement in Pompe disease.

### EXAMPLES

While the following examples describe specific embodiments, it is understood that variations and modifications will occur to those skilled in the art. Accordingly, only such limitations as appear in the claims should be placed on the invention.

### Example 1

This example compares various doses of systemic delivery of GAA gene therapy with liver-directed GAA gene therapy.

### Materials and Methods

Animal Models, Experimental design, Treatment, and Tissue Processing

Two animal models of Pompe disease were used in the study: a GAA−/− knockout (KO) strain carrying a targeted deletion of exon 6 (31) and muscle-specific autophagy-deficient GAA−/− (MLCcre: Atg7F/F: GAA−/−; referred to as DKO) strain, in which a critical autophagy gene, ATG7, was excised in skeletal muscle by Cre recombinase (48).

Two viral vectors, AV9.CAG.BiP.vIGF2.hGAAco(V810I).rBG.KanR (Systemic; SYS) and AAV9.TBG.Sp7.delta8hGAA(V763)co.rBG (Liver-directed; LD) were used at three vector dosages: High (5×10\hat{ }13 vg/kg), intermediate (2.5×10\hat{ }13 vg/kg), and low (0.5×10\hat{ }13 vg/kg).

Male KO mice (n=63) were randomly assigned to three groups. Group 1 (high dose/short-term/young) included young (3.5-month-old) KO mice receiving high doses of SYS (n=3) or LD vector (n=3); muscle tissues were analyzed after one month post injection. Group 2 included both young (3-month-old) and old (8-9-month-old) KO mice receiving intermediate doses of SYS or LD vector. Young KO mice were treated (n=11 with SYS; n=8 with LD) for a period of one month (intermediate dose/short-term/young) and 7 months (n=3 with SYS; n=3 with LD; intermediate dose/long-term/young). Old KO mice were treated for a period of 7-8 months (n=8 with SYS; n=6 with LD; intermediate dose/long-term/old). Heart and diaphragm (n=3 with SYS only) were analyzed after short-term treatment of young KO; brain samples were analyzed after long-term treatment of young (SYS n=3; LD n=3) and old (SYS n=7; LD n=4) KO mice. Group 3 included young KO mice receiving low doses of SYS or LD vectors; these mice were treated for a period of one month (n=5 with SYS; n=4 with LD; low dose/short-term/young) and 6 months (n=4 with SYS; n=5 with LD; low dose/long-term/young). DKO mice (n=6) were used for the evaluation of the effect of SYS vector (low dose/short-term/young).

TABLE 5
Experimental design

| | Group 1 | Group 2 | | | Group 3 | |
|---|---|---|---|---|---|---|
| | High | Intermediate* | | | Low | |
| Dosage | 5 × 10\hat{ }13 | 2.5 × 10\hat{ }13 | | | 0.5 × 10\hat{ }13 | |
| Age at | 3.5 mo | 3 mo | | 8-9 mo | 3.5 mo | 3 mo |
| start | | | | | | |
| Duration | 1 mo | 1 mo | 7 mo | 7-8 mo | 1 mo | 6 mo |
| of therapy | | | | | | |
| Number of | n = 3 SYS | n = 11 SYS | n = 3 SYS | n = 8 SYS | n = 5 SYS | n = 4 SYS |
| animals | n = 3 LD | n = 8 LD | n = 3 LD | n = 6 LD | n = 4 LD | n = 5 LD |
| (total) | | | | | | |
| Tissues | Muscle | Muscle n = 8 | Muscle | Muscle | Muscle | Muscle |
| analyzed | | Heart & | Brain | Brain | | |
| | | Diaphragm | n = 3 SYS; | n = 7 SYS; | | |
| | | n = 3 SYS | n = 3 LD | n = 4 LD | | |

Total number of SYS-treated KO: n = 34; Total number of LD-treated KO: n = 29
Total number of WT: n = 25 (4-4.5-mo-old n = 11; 10-15.5-mo-old n = 14)
Total number of untreated GAA-KO: n = 30 (4-4.5-mo-old n = 18; 10-15.5-mo-old n = 12)
*In addition, intermediate dosage was used in 3-month-old DKO mice (n = 6); DKO untreated (n = 3)

All animals were treated with a single injection of either vector in the tail vein (study day 0). Age matched WT (n=25) and untreated KO mice (n=30) were used as controls. Most WT and untreated KO mice were males; however, females were also used in some experiments since we didn't see any difference in the levels of GAA activity, glycogen content, and markers of autophagy between the sexes in these strains.

The resected tissues were either immediately snap-frozen in Isopentane/Liquid nitrogen and stored at −80° C. until use for biochemical analyses or fixed for Periodic acid-Schiff (PAS) stain and immunostaining of single muscle fibers.

### Measurement of GAA Activity and Glycogen Levels

GAA activity was measured by using 4-methylumbelliferyl-α-D-glucoside (4 MUG; Sigma-Aldrich) as the artificial fluorogenic GAA substrate as described (88) Briefly, muscle samples were homogenized in RIPA buffer (1 mg/20 L water), sonicated, and centrifuged at 16,000 g at 4° C. for 15 min. The supernatants were diluted in distilled water; the diluted supernatants (10 L) were incubated with the substrate (20 L) in 0.2 M sodium acetate buffer (pH 4.3) for 1 h at 37° C.; the enzymatic reaction was stopped by adding 0.5 M carbonate buffer (pH 10.5). 4-Methylumbelliferone (Sigma-Aldrich) was used as a standard. Fluorescence was measured on a multi-label plate reader (TECAN, SPARK 10M) at 350 nm excitation/460 nm emission. The GAA activity was expressed as nmol of 4-MU released/hour/per mg of protein. Protein concentration (BCA assay) was measured and used to normalize the data.

Glycogen content was measured as the amount of glucose released after glycogen digestion with Aspergillus Niger amyloglucosidase (Sigma-Aldrich) as described (89). Briefly, the diluted tissue lysates were denatured at 100° C. for 3 min, centrifuged at 9,000 RPM at room temperature for 3 minutes, and the supernatants were incubated with/or without 0.175 U/mL amyloglucosidase for 90 min at 37° C. in 0.1 M potassium acetate buffer (pH 5.5) and boiled again to stop the reaction. The released glucose was measured using Glucose (Hexokinase) Liquid Reagents (Fisher) as recommended by the manufacturer; the absorbance at 340 nm was read on the Agilent Technologies Cary 60 UV-VIS Spectrophotometer.

### Western Blot Analysis

For Western blotting, skeletal muscle [superficial (pale) part of gastrocnemius muscle], cardiac muscle, the diaphragm, and brain tissues were used for analyses. The tissues were homogenized in RIPA buffer (PBS containing 1% NP-40, 0.5% sodium deoxycholate, 0.1% SDS, and a protease/phosphatase inhibitor cocktail), and centrifuged for 15 min at 16,000×g at 4° C. Protein concentrations of the supernatants were measured using the Bio-Rad Protein Assay (Bio-Rad Laboratories, Inc.), and equal amounts of protein were loaded on NuPAGE Bis-Tris protein gels (Thermo Fisher Scientific,) in denaturing condition. Separated proteins were electro-transferred onto nitrocellulose membranes (Invitrogen, Carlsbad, CA, USA). Membranes were then treated with blocking buffer (5% nonfat milk), incubated with primary antibodies overnight at 4° C., washed, incubated with the appropriate HRP-linked secondary antibodies and washed again. Horseradish peroxidase (HRP)-chemiluminescence was developed using Azure Radiance plus kit and scanned on imager (Azure biosystems). The following primary antibodies (all diluted 1:1000) were used: total- and phosphorylated AMPK (#2535; T¹⁷² #5831; rabbit monoclonal), total- and non-phosphorylated 4EBP1 (#4923; T⁴⁶ #9644, rabbit monoclonal) were purchased from Cell Signaling Technology; LAMP1 (CD107a #553792; rat monoclonal; BD Transduction Laboratories); GAA (#ab137068; rabbit monoclonal), SQSTM1/p62 (#ab56416; mouse monoclonal) and Glyceraldehyde-3-phosphate dehydrogenase (GAPDH; #ab9485; rabbit polyclonal) were purchased from Abcam; Galectin-3 (#sc-32790; mouse monoclonal) were from Santa Cruz Biotechnology, and LC3B (#L7543; rabbit polyclonal) were purchased from Sigma Aldrich.

### Immunostaining of Single Muscle Fibers

Superficial (pale) part of gastrocnemius muscle was used for single muscle fibers analysis. Muscle fixation, isolation of single fibers, and immunostaining were performed as described (90) with some modifications. Briefly, muscle strips were fixed with 2% paraformaldehyde (Electron Microscopy Science) in 0.1 M phosphate buffer for 30-40 minutes at room temperature; the strips were pinned at their resting length on a Sylgard plate. The strips were then removed from the plate, washed with PBS and incubated in cold methanol for 6 min at −20° C. The samples were then rinsed again with PBS and placed in 0.04% saponin in PBS on a Sylgard plate for manual isolation of single fibers under a dissecting microscope. For longer storage, muscle strips were transferred to 12.5%, then 25% and 50% glycerol in PBS and stored at −20° C. The isolated fibers were stained with LAMP1 (CD107a #553792; 1:200; rat monoclonal; BD Transduction Laboratories) and LC3 (LC3B #L7543; 1:500; rabbit polyclonal; Sigma Aldrich) antibodies using M.O.M. kit (Vector Laboratories, Burlingame, CA, USA). For each immunostaining and for confocal analysis, at least 25-30 fibers were isolated. The images were captured using a Zeiss LSM 780 confocal microscope under the Zeiss Efficient Navigation (ZEN) software.

### Histology

For PAS staining, muscle tissues were fixed in 3% glutaraldehyde (EM grade, Electron Microscopy Sciences, Hatfield, PA) in 0.2 M sodium cacodylate buffer for 4 hours at 4° C.; the samples were then washed in 0.1 M sodium cacodylate buffer and stored at 4° C. in the same buffer. For PAS staining of brain tissues, the samples were fixed in 10% neutral-buffered formalin (NBF) for 48 hours. The tissues were then postfixed in neutral-buffered formalin containing 1% periodic acid for another 48 hours at 4° C., embedded in paraffin, sectioned, and stained with PAS by standard procedures. All stained slides were scanned and captured using a Hamamatsu NanoZoomer slide-scanner; the images were viewed and captured using NDP.view2 software from Hamamatsu.

### Functional Muscle Strength Test

Muscle strength test was performed using a Grip Strength Meter (Columbus Instruments, Columbus, OH) according to the manufacturer's recommendations. The test measures maximal combined forelimbs/hindlimbs grip strength as an indicator of neuromuscular function. Briefly, the animal is placed on a wire grid and allowed to grasp it; the animal's base of the tail is then gently pulled backward horizontally until the grasp is released. The strength is measured by the grasping applied by the mouse on a grid that is connected to a monitoring device. The force applied to the grid is recorded as the maximal peak force displayed by the animal. Each mouse was tested three times with 10-15 min intervals per session; three sessions were conducted over three consecutive days.

### Statistics

Statistical significance was calculated by using GraphPad Prism software. One-way ANOVA and unpaired two-tailed Student's t test were performed. Data presented as mean±SD; *p<0.05; **p<0.01; ***p<0.001; ****p<0.0001).

### Results

Two viral vectors (Amicus Therapeutics) were used: 1) AAV9.CAG.BiP.vIGF2.hGAAco(V810I).rBG.KanR (Systemic; SYS) vector is driven by the CAG promoter [cytomegalovirus (CMV) early enhancer/chicken 3 actin] to achieve systemic distribution among multiple tissues including CNS through transduction and cross-correction, and 2) AAV9.TBG.Sp7.delta8hGAA(V763)co.rBG (Liver-directed; LD) vector is driven by human thyroxine binding globulin(TBG) promoter to direct efficient transgene expression in the liver. The in vivo efficacy of the vectors was evaluated following a single intravenous administration in a GAA KO model of Pompe disease (31) (thereafter referred to as KO) at 3-3.5 months (young) or 8-9 months (old) of age. The animals were treated with either SYS or LD vector at doses of 5×10\hat{ }13 vg/kg (high dose), 0.5×10\hat{ }13 vg/kg (low dose), or 2.5×10\hat{ }13 vg/kg (intermediate dose). The tissues were harvested following 1 month (short term) or 6-8 months (long term) after dosing. The experimental design and the number of animals is shown in Table 1. Considering the reported sex bias for liver expressed AAV vectors in mice, i.e., much higher transduction efficiency in the liver of male compared to female mice, regardless of mouse strain, cDNA or the promoter (32, 33), only KO males were used for the experiments to avoid variability in liver transduction.

### Both Systemic and Liver-Directed Gene Transfer Reverse Muscle Pathology at a High Vector Dose

Three-month-old KO were treated with ether SYS or LD vector at a dose of 5×10\hat{ }13 vg/kg, and skeletal muscle (gastrocnemius) was analyzed after one month on therapy. Western blot analyses showed greater amount of mature lysosomal form of hGAA protein and higher enzyme activity in muscle tissues from SYS-treated compared to LD-treated KO mice. GAA activity exceeded the WT values with both treatments, and regardless of the difference, both therapies reduced muscle glycogen content to near normal levels.

Next, we evaluated the effect on autophagy—a major secondary abnormality in the diseased muscle (34) [reviewed in (35, 36)]—by measuring the levels of lysosomal-autophagosomal markers and by confocal microscopy of immunostained isolated single muscle fibers. Both treatments normalized the levels of LAMP1 (lysosomal marker), LC3 (autophagosomal marker (37), and the autophagy-specific substrate SQSTM1/p62 (38, 39). In addition, we have looked at the level of galectin 3 (LGALS3), a marker of damaged lysosome (40-42); we have previously reported its positive association with autophagic buildup in the diseased muscle (43). Consistent with our previous data, the level of galectin 3 was markedly increased in KO muscle, and this increase was fully reversed following the treatments. Immunostaining of single muscle fibers with LAMP1 and LC3 showed enlarged lysosomes throughout the fibers and LAMP1/LC3-positive areas of autophagic accumulation in the core of myofibers in KO, as is typical for Pompe disease (35); in contrast, the vast majority of fibers from treated KO appear normal with small dot-like LAM1/LC3 positive structures, often located next to each other.

We have also examined the effect of gene therapies on two major lysosome-linked signaling pathways, which reciprocally and antagonistically control autophagy—the energy sensor AMP-activated protein kinase (AMPK) and the growth-promoting mammalian target of rapamycin complex 1 (mTORC1). We have previously reported activation of AMPK and diminished mTORC1 activity in muscle from KO mice; this inference was drawn from systematic analysis of the upstream inputs and the downstream targets of these kinases (43, 44, 45). Here, we limited the analysis to the components that reflect the changes in AMPK and mTORC1 activity. Consistent with our previous data, p-AMPK^{Thr172}/AMPK ratio and the level of non-phosphorylated 4EBP1 (the eukaryotic initiation factor 4E binding protein; a direct mTORC1 substrate) were significantly increased in KO. In its non-phosphorylated state, 4EBP1 binds the translation initiation factor eIF4E, thereby inhibiting 5′-cap-dependent translation (46) by competing with eIF4G and blocking the formation of the active eIF4E/eIF4G complex (37). Both treatments normalized p-AMPK^{Thr172}/AMPK ratio; the levels of non-phosphorylated 4EBP1 fell significantly and reached the WT levels following SYS gene transfer, whereas the values tended to decrease after LD gene transfer. Thus, both treatments at high dosages completely reversed lysosomal glycogen accumulation, eliminated autophagic buildup, and improved AMPKmnTORC1 signaling after one month of therapy.

### Neither Systemic Nor Liver-Directed Gene Transfer Provides Full Correction of Muscle Pathology at a Low Vector Dose

The same pattern of greater amount of lysosomal GAA protein and higher GAA activity with SYS compared to LD gene transfer was observed after low dose (0.5×10\hat{ }13 vg/kg) short-term treatment. However, the higher GAA activity with SYS gene transfer didn't reach physiological levels measured in WT muscle and resulted in a modest, albeit significant, glycogen reduction compared to untreated KO. In contrast, no reduction in muscle glycogen was observed following LD gene transfer. Periodic acid-Schiff (PAS) staining for glycogen showed abundant PAS-positive structures in every fiber from KO and LD-treated KO mice, whereas a number of fibers from SYS-treated animals appeared completely clear from excess glycogen, in agreement with the results of biochemical measurement of glycogen content.

No significant changes in the levels of LAMP1, LC3, and SQSTM1/p62 were observed with either treatment when compared to those in untreated KO, but immunostaining of single muscle fibers with LAMP1 and LC3 revealed a modest but statistically significant difference in the number of fibers with autophagic buildup: 99% (n=159) in LD-treated and 88% (n=126) in SYS-treated mice.

We have also explored the therapeutic efficacy of low dose short-term SYS gene transfer in the previously described muscle-specific autophagy-deficient KO mice (MLCcre:Atg7F/F:GAA−/−, referred to as DKO), in which glycogen accumulation in muscle is markedly reduced compared to KO (48). The LC3-II [an autophagosome membrane-bound LC3 form (37)] was completely absent in muscle lysates from DKO mice, thereby confirming suppression of autophagy. Similar to what was observed in SYS-treated KO, GAA activity did not reach physiological levels in DKO. However, these low levels of enzyme activity resulted in further glycogen reduction bringing muscle glycogen content close to normal levels. Consistent with these data, PAS staining showed multiple normal or near normal myofibers, underscoring the benefit of autophagy modulation to achieve therapeutic significance at lower vector doses.

Low dose long-term (6 months) treatments with SYS or LD gene transfer did not rescue muscle pathology but the outcomes favored SYS over LD therapy. The same pattern of greater amount of lysosomal GAA (and higher enzyme activity without reaching the WT values) in muscle from SYS-treated compared to LD-treated KO was also observed after 6 months after dosing. However, the continuously expressed GAA protein and the ongoing process of cross-correction did lead to a significant decrease in the level of LAMP1 and in muscle glycogen content in SYS-treated KO compared to age-matched 9-month-old untreated KO; consequently, PAS staining of muscle samples showed multiple glycogen-free fibers. In contrast, glycogen content in muscle from LD-treated KO fell only marginally (without reaching statistical significance compared to untreated KO), and the vast majority of fibers were still filled with PAS-positive structures. Thus, LD gene transfer at a low dose had a negligible effect on muscle pathology, whereas SYS gene transfer showed a partial but significant improvement, particularly in the long-term.

### Systemic Gene Transfer Provides Superior Rescue of Muscle Pathology Compared to Liver-Directed at an Intermediate Vector Dose

Short-term treatment of young (3-month-old) KO animals at a dose of 2.5×10\hat{ }13 vg/kg revealed a striking difference between the two approaches. All outcome measures were superior in SYS-treated mice: greater amount of lysosomal GAA protein and supraphysiological GAA activity; more efficient glycogen clearance; a larger reduction in the levels of autophagosomal-lysosomal markers (all reaching the WT levels, except for p62 which was significantly reduced) and proteins involved in mTORC1/AMPK signaling.

These results are further supported by histological analysis of muscle sample and by LAMP1/LC3 immunostaining of muscle fibers. PAS-stained sections from SYS-treated mice were indistinguishable from the WT, and the vast majority of myofibers [88%; n=87 fibers as opposed to only ~30% with LD therapy (n=153)] were free from autophagic. Although the outcome measures improved on LD therapy, the effect was much weaker. Furthermore, SYS gene transfer resulted in a significant increase in muscle fiber size (at least 60 fibers were analyzed for each condition) and improvement of muscle strength after just one month on therapy.

These compelling short-term results of SYS gene transfer prompted us to look at the two other critical therapeutic targets-cardiac muscle and diaphragm-under the same experimental conditions; diaphragmatic dysfunction is a main contributor to respiratory insufficiency in Pompe disease (49). Lysosomal GAA protein was easily detectable in the heart and diaphragm; both tissues had supraphysiological levels of GAA activity leading to complete glycogen clearance from the heart and near complete clearance from the diaphragm, as shown by biochemical assay and PAS staining for glycogen.

The benefits of short-term SYS gene transfer were maintained 7 months after dosing, as indicated by the abundance of GAA protein, the enzyme activity, and muscle glycogen clearance. Furthermore, long-term therapy fully rescued autophagic pathology (none of the LAMP1/LC3 stained fibers contained autophagic buildup (n=73), normalized muscle fiber size, and restored muscle function. In contrast to what was observed in the short-term, LD gene transfer also showed major improvements 6 months after dosing, and the difference between the two approached became much more subtle. Although the amount of GAA protein and GAA activity were invariably lower than with SYS gene transfer, muscle glycogen levels were not significantly different between the two groups. Histological analysis revealed overall efficient glycogen clearance, but multiple fibers and groups of fibers contained small PAS-positive structures. The enlarged LAMP1-positive lysosomes were also detected by immunostaining, and occasional (3-4%) fibers (n=62) were profoundly atrophic, most likely contributing to incomplete restoration of muscle function.

We have also investigated long-term (7-8 months) effects of the two vectors in old (8-9 months) KO mice. At the start of therapy, these mice show obvious clinical signs of muscle weakness and waddling gait (31, 50); the condition deteriorates progressively, and by the age of 13-14 months the animals drag hind limbs and display profoundly wasted lower back muscle and kyphosis necessitating euthanasia according to the guidelines. Both treatments dramatically changed the natural course of the disease, and at the end of the follow-up period (at the age of 15-16 months) the KO appeared healthy, did not clench hind limbs to the body when suspended by the tail, and were able to stand on their hind limbs.

As in all the experiments described above, the amount of lysosomal GAA and the enzyme activity were significantly higher with SYS compared to LD gene transfer. However, all other outcomes—muscle glycogen content and the levels of lysosomal/autophagic markers were similar between the two groups. A closer look at PAS-stained sections of muscle biopsies revealed the presence of PAS-positive structures and autophagic buildup in samples from LD-treated but not SYS-treated mice. This finding was supported by immunostaining of isolated muscle fibers with LAMP1/LC3: autophagic buildup was present in 16% of fibers (n=102) from LD-treated mice but in only ~1.4% (1 of 69) of fibers from SYS-treated KO. There was a significant increase in grip strength by both treatments, more so by SYS gene transfer. Thus, while SYS gene transfer at the intermediate dose had superior outcomes in the short-term, over time LD therapy appeared almost—but not quite—as efficacious.

In addition, we have compared GAA activity and the degree of muscle glycogen clearance in all sets of experiments. Overall, the correlation between the two appears to be fairly strong. This comparison allowed us to address the critical question of the required level of enzymatic activity to ensure a complete reversal of pathology. The data indicate that muscle glycogen returns to normal, and the pathology is fully rescued only when the levels of GAA activity exceed the upper limit of the WT range. However, when GAA activity is below WT and even within the WT range, the reversal is not complete. SYS gene transfer at high and intermediate dosages resulted in the levels of enzyme activity far exceeding the WT range, and the therapy was remarkably efficient in reversing glycogen storage as well as multiple downstream abnormalities stemming from lysosomal enlargement.

### Systemic Gene Transfer Provides Superior Rescue of Brain Pathology Compared to Liver-Directed at an Intermediate Vector Dose

Glycogen accumulation in the central nervous system (CNS) has been shown to contribute to respiratory insufficiency and skeletal muscle dysfunction (51-54). Accumulating evidence indicates that glycogen storage in brain tissue is associated with signs of neurological deficit, most prominent in ERT-treated long-term survivors with IOPD (12, 13, 55). Age-dependent glycogen storage in multiple regions of the brain in KO mice is also well-documented (50, 56).

Brain tissues from long-term treated young (3 mo of age) and old (8-9 mo of age) KO mice receiving intermediate dose of the AAV vectors were used for analysis. Western blot showed that the amount of lysosomal GAA protein, which possesses optimal glycogen hydrolyzing activity, was greater in SYS- compared to LD-treated KO (similar to what was observed in muscle), but the enzyme activity was not different between the two groups. This apparent discrepancy is likely due to the abundance of the GAA precursor protein, which may contribute to the enzymatic activity in the brain of LD-treated KO (16, 57). In fact, the 110 kDa precursor was always detected in muscle on western blots in all sets of the experiments from LD-treated mice; however, its abundance relative to the amount of lysosomal GAA was not as prominent as in the brain tissue. These data suggest that the liver secreted circulating GAA precursor leaks across the BBB but its uptake and processing into mature lysosomal form is very limited in brain tissue. This is supported by only a modest glycogen reduction (~30%) in young and a slight tendency to decrease (insignificant) in old LD-treated mice.

In contrast, SYS gene transfer resulted in near complete glycogen clearance in both young and old animals. Consequently, PAS staining revealed similarity between the samples from WT and SYS treated animals, whereas brain sections from KO and LD-treated mice showed glycogen storage in neurons and glial cells.

### Discussion

Unlike most other lysosomal storage disorders, Pompe disease is not associated with prominent neurological symptoms. Therefore, the disease has historically been viewed as an inherited metabolic myopathy, and the therapeutic efforts focused primarily on correcting skeletal and cardiac muscle abnormalities. Given the limited therapeutic efficacy of ERT in rescuing skeletal muscle pathology, multiple preclinical studies explored gene therapy using AAV vectors of different serotypes expressing GAA to deliver the transgene to skeletal muscle either by local or systemic route [reviewed in (22, 23, 25)]. The safety of the local (intradiaphragmatic) delivery was supported by the first clinical trial designed to improve respiratory insufficiency in ERT-treated patients with IOPD (28, 29). A recent preclinical study involved systemic delivery of an AAV8 vector expressing GAA protein under the control of a muscle specific promoter/enhancer (AT845). Complete glycogen clearance in the heart and quadriceps was achieved at a vector dose of 3×10\hat{ }14 vg/kg three months after administration into young 10-11-week-old KO mice (58). AT845 is currently being tested in a Phase I/II clinical trial (NCT04174105; Astellas Gene Therapies). However, the paradigm shift in Pompe disease—which is nowadays recognized as a neuromuscular disorder—underscores the limitation of this approach: the design of the transgene provides muscle-restricted expression of GAA protein (58). The central nervous system (CNS), along with cardiac and skeletal muscles, is emerging as a new therapeutic target in Pompe disease [reviewed in (54, 59)].

An alternative strategy based on converting the liver into a biofactory for production and secretion of GAA at least partially addresses the problem. The newly synthesized secreted enzyme is recaptured by various organs and tissues throughout the body in a process called ‘cross-correction’ (60-63). This approach has gained increasing attention following the generation of AAV vectors containing a hepatocyte-specific promoter (human alpha 1-antitrypsin; hAAT) and an engineered GAA transgene encoding for GAA protein with enhanced secretion, thus providing sustained high levels of the enzyme in the circulation (AAV8-secGAA) (64, 65).

The blood-brain barrier (BBB), which is thought to be impermeable to lysosomal enzymes (except in the newborn period), was shown to be at least partially breached when high plasma activity of the enzymes was attained; amelioration of CNS pathology was observed in a mucopolysaccharidosis type IIIA (MPSIIIA; a deficiency of sulfamidase) mouse model following AAV-mediated liver-directed gene transfer (66). Indeed, liver-targeted gene transfer of secretable GAA (AAV8-secGAA) in both young and old KO mice resulted not only in efficient glycogen clearance in cardiac and skeletal muscles, but also in partial clearance in the CNS at a vector dose of 2×10 vg/kg (64, 67). These results were further confirmed in a new immunodeficient model of Pompe disease (GAA^{-/-}Cd4^{-/-}) (68). Liver-directed gene therapy is now being tested in the ongoing clinical trial (NCT04093349; SPK-3006; Spark Therapeutics).

In this study, we have used an AAV9-based liver-directed vector expressing a version of secretable GAA driven by a liver-specific human thyroxine binding globulin promoter (TBG); the vector was designed to mimic the effect of AAV8-secGAA. Indeed, our findings mostly corroborate the published data obtained with AAV8-secGAA in that we too observed rescue of muscle pathology and a modest decrease in the brain glycogen storage (~30%) after AAV9-mediated long-term liver-targeted gene transfer. However, the effect was observed at a much higher vector dose of 2.5×10—vg/kg compared to the one used for AAV8-secGAA.

We have also asked whether it is possible to develop a treatment that could provide more efficient glycogen reduction in the CNS while preserving (or even improving) the ability to rescue muscle pathology. We have shown that systemic delivery of an AAV9 vector containing IGF2-tagged GAA driven by a ubiquitous promoter can achieve this goal. AAV9 serotype has been shown to robustly transduce multiple tissues, including cardiac and skeletal muscle, the diaphragm, and the brain [reviewed in (69)]—all of which are therapeutic targets in Pompe disease.

Unlike liver-directed, long-term systemic gene transfer at the same dose of 2.5×10\hat{ }13 vg/kg resulted in complete or near complete clearance of glycogen in the brain of young and old KO mice, as shown by biochemical assay and histological analysis. This striking difference between the two gene therapy approaches indicates that systemic approach has a clear advantage over liver-targeted therapy in correcting the CNS pathology.

The effect of systemic delivery in skeletal muscle was equally impressive—even in old severely affected KO mice, long-term therapy reduced muscle glycogen to the WT levels, fully reversed autophagic defect, normalized the size of the myofibers, and restored muscle function. In fact, long-term systemic gene transfer proved somewhat superior to liver-directed treatment. Residual lysosomal glycogen storage, atrophic fibers, and fibers with autophagic buildup could still be detected after liver-directed therapy, but not after systemic gene transfer. Even at a dose of 0.5×10\hat{ }13 vg/kg, which was not sufficient to reach therapeutic significance with either vector, the outcome of systemic delivery was slightly better compared to liver-directed treatment: Muscle glycogen burden and the numbers of fibers with autophagic buildup were significantly reduced after systemic gene transfer, whereas the effect of liver-directed therapy was negligible.

The most dramatic difference between the two vectors, in terms of their effect in skeletal muscle, was observed in young 3-month-old KO mice after short-term therapy at a dose of 2.5×10\hat{ }13 vg/kg. Systemic gene transfer fully rescued muscle pathology—including excessive glycogen accumulation, autophagic defect, and mTORC1/AMPK signaling—just one month after the start of therapy. Importantly, these results were achieved using skeletal muscle (superficial pale part of gastrocnemius) which is most refractory to therapy (34, 70). To the best of our knowledge, this outcome has never been achieved in such a short time with any therapy for Pompe disease. In contrast, liver-directed gene transfer required much longer to fully exert its effect in skeletal muscle; this observation is in agreement with published data showing that hepatic expression of GAA and muscle exposure to hepatocyte-secreted GAA are time-dependent (68). The importance of rapid response following systemic gene transfer cannot be overstated—shortening the time to halt the progression of the disease is critical to avert further age-dependent muscle deterioration. Efficient glycogen clearance was also observed in the heart and diaphragm after one month of systemic therapy.

Two cellular mechanisms account for the therapeutic efficacy of systemic gene transfer in KO mice: 1) high tropism of AAV9 toward muscle and its ability to cross the BBB, thus enabling efficient transduction of the target tissues, and 2) the transgenic expression of a chimeric IGF2-tagged GAA with high affinity for the bifunctional IGF2/CI-MPR, thus allowing for efficient cross-correction of the surrounding non-transduced cells within the tissue itself.

It has been demonstrated that a short IGF2 fragment linked to the therapeutic lysosomal enzyme can provide a high affinity ligand for CI-MPR through its interaction with the IGF2 binding domain that is different than mannose-6-phosphate (M6P) binding sites (14, 30). M6P-capped ligands bind the sites in repeats 3, 5, and 9 within the 15 homologous repeats on the extracytoplasmic region of the CI-MPR receptor, whereas high affinity IGF2 binding sites are located in repeats 11 and 13 (71, 72).

The benefits of the IGF2 peptide-based glycosylation-independent lysosomal targeting (GILT) strategy for enzyme replacement therapy (ERT) were first shown in a model of mucopolysaccharidosis type VII (MPS VII) (30). Since then, this approach has been explored in several lysosomal storage disorders by generating IGF2-tagged recombinant enzymes for ERT or by incorporating this peptide tag into the viral cassettes for gene therapy.

IGF2-tagged lysosomal alpha-N-acetylglucosaminidase (NAGLU), the enzyme that is deficient in a severe neurodegenerative mucopolysaccharidosis type IIIB (Sanfilippo syndrome type B), was shown to normalize the enzyme activity in primary rat-derived neurons and astrocytes and in patients' fibroblasts, whereas untagged rhNAGLU only normalized NAGLU activity in microglia (73). As for Pompe disease, targeted intralingual administration of an AAV9 encoding IGF2-tagged GAA (AAV9-DES-IGFIIcoGAA) resulted in efficient glycogen clearance and reversal of pathology in lingual myofibers and hypoglossal (XII) motoneurons in a KO model (74). Lingual weakness and hypoglossal neuropathology are common characteristics of Pompe disease (75-77). GILT-tagged GAA is currently being investigated in the context of lentiviral-mediated hematopoietic stem/progenitor cells (HSPCs) gene therapy for Pompe disease (78). The potential of ex vivo HSPC-mediated lentiviral gene therapy for Pompe disease lies in its capacity to correct both peripheral tissues and the CNS, as well as to serve as an immunomodulatory treatment to overcome immune challenges to ERT (79-81).

Furthermore, a chimeric GILT-tagged human recombinant GAA (Reveglucosidase alfa; BMN 701) for ERT was shown to reduce muscle glycogen deposits and improve respiratory function much more efficiently than untagged recombinant human GAA in KO mice (14) (82). Of note, the pharmacokinetics, safety, and exploratory efficacy of Reveglucosidase alfa were assessed in a Phase I/II clinical trial in ERT naïve patients with late-onset Pompe disease. The patients showed improvement in respiratory function and mobility after 72 weeks of treatment (83). However, a small subset of patients developed symptomatic hypoglycemia, which was attributed to a pharmacologic effect of the IGF2 moiety that can bind IGF1 and insulin receptors with low affinity. Importantly, in this study we used a version of IGF2 peptide (Amicus proprietary) with strategic amino acid substitutions to eliminate off target binding.

The amount of fully active mature form of GAA and the enzyme activity in skeletal muscle in all experimental settings were invariably higher with systemic compared to liver-directed gene transfer—regardless of the dosages, animal age, and the duration of therapy—suggesting a superior level of cellular uptake and cross-correction. Although GAA activity is not necessarily considered a good predictor of glycogen clearance (68, 84, 85), we did see a good correlation between the two when the enzyme activity exceeded the physiological levels, thus establishing a threshold of GAA activity that is required to reverse the phenotype. Analyses of the whole muscle, such as commonly used gastrocnemius, quadriceps, and triceps, may have skewed the data because these muscles are composed of mixed fiber types that respond differently to therapy. As mentioned above, we routinely use the superficial (pale) part of gastrocnemius muscle, which is more homogeneous and consists of predominantly type IIB myofibers. Notably, even at the highest vector dose when the GAA activity reached supraphysiological levels, no deleterious effect was observed in skeletal muscle, and glycogen reduction didn't drop below physiological levels.

Taken together, the data indicate that a single systemic administration of AAV9 vector carrying chimeric IGF2-tagged GAA can rapidly and efficiently rescue the pathology in all key therapeutic targets in Pompe disease, including the CNS.

All documents referred to in this application are hereby incorporated by reference in their entirety, including the references mentioned above and the following references:
1. Van der Ploeg A T, and Reuser A J. Pompe's disease. Lancet. 2008; 372(9646):1342-53.
2. Reuser J J, Hirschhorn, R., Kroos M. A. Pompe Disease: Glycogen Storage Disease Type II, Acid α-Glucosidase (Acid Maltase) Deficiency. The Online Metabolic and Molecular Bases of Inherited Disease Lysosomal Storage Disorders, A L Beaudet, B Vogelstein, K W Kinzler, S E 796 Antonarakis, A Ballabio, K M Gibson, and G Mitchell, eds (The McGraw-Hill Companies, Inc). 2018.
3. Kishnani P S, Hwu W L, Mandel H, Nicolino M, Yong F, and Corzo D. A retrospective, multinational, multicenter study on the natural history of infantile-onset Pompe disease. J Pediatr. 2006; 148(5):671-6.
4. van der Beek N A, de Vries J M, Hagemans M L, Hop W C, Kroos M A, Wokke J H, et al. Clinical features and predictors for disease natural progression in adults with Pompe disease: a nationwide prospective observational study. Orphanet J Rare Dis. 2012; 7:88.
5. Kishnani P S, Corzo D, Nicolino M, Byrne B, Mandel H, Hwu W L, et al. Recombinant human acid [alpha]-glucosidase: major clinical benefits in infantile-onset Pompe disease. Neurology. 2007; 68(2):99-109.
6. Kishnani P S, Corzo D, Leslie N D, Gruskin D, Van der P A, Clancy J P, et al. Early treatment with alglucosidase alpha prolongs long-term survival of infants with Pompe disease. Pediatr Res. 2009; 66(3):329-35.
7. Nicolino M, Byrne B, Wraith J E, Leslie N, Mandel H, Freyer D R, et al. Clinical outcomes after long-term treatment with alglucosidase alfa in infants and children with advanced Pompe disease. Genet Med. 2009; 11(3):210-9.
8. Chakrapani A, Vellodi A, Robinson P, Jones S, and Wraith J E. Treatment of infantile Pompe disease with alglucosidase alpha: the U K experience. Journal of inherited metabolic disease. 2010; 33(6):747-50.
9. Harlaar L, Hogrel J Y, Perniconi B, Kruijshaar M E, Rizopoulos D, Taouagh N, et al. Large variation in effects during 10 years of enzyme therapy in adults with Pompe disease. Neurology. 2019; 93(19):e1756-e67.
10. Gutschmidt K, Musumeci O, Diaz-Manera J, Chien Y H, Knop K C, Wenninger S, et al. STIG study: real-world data of long-term outcomes of adults with Pompe disease under enzyme replacement therapy with alglucosidase alfa. J Neurol. 2021; 268(7):2482-92.
11. Prater S N, Banugaria S G, DeArmey S M, Botha E G, Stege E M, Case L E, et al. The emerging phenotype of long-term survivors with infantile Pompe disease. Genetics in medicine: official journal of the American College of Medical Genetics. 2012; 14(9):800-10.
12. Ebbink B J, Poelman E, Plug I, Lequin M H, van Doom P A, Aarsen F K, et al. Cognitive decline in classic infantile Pompe disease: An underacknowledged challenge. Neurology. 2016; 86(13):1260-1.
13. Ebbink B J, Poelman E, Aarsen F K, Plug I, Regal L, Muentjes C, et al. Classic infantile Pompe patients approaching adulthood: a cohort study on consequences for the brain. Dev Med Child Neurol. 2018; 60(6):579-86.
14. Maga J A, Zhou J, Kambampati R, Peng S, Wang X, Bohnsack R N, et al. Glycosylation-independent lysosomal targeting of acid alpha-glucosidase enhances muscle glycogen clearance in Pompe mice. The Journal of Biological Chemistry. 2013; 288:1428-38.
15. Do H V, Khanna R, and Gotschall R. Challenges in treating Pompe disease: an industry perspective. Ann Transl Med. 2019; 7(13):291.
16. Selvan N, Mehta N, Venkateswaran S, Brignol N, Graziano M, Sheikh M O, et al. Endolysosomal N-glycan processing is critical to attain the most active form of the enzyme acid alpha-glucosidase. J Biol Chem. 2021; 296:100769.
17. Kishnani P S, Goldenberg P C, DeArmey S L, Heller J, Benjamin D, Young S, et al. Cross-reactive immunologic material status affects treatment outcomes in Pompe disease infants. MolGenetMetab. 2010; 99(1):26-33.
18. Diaz-Manera J, Kishnani P S, Kushlaf H, Ladha S, Mozaffar T, Straub V, et al. Safety and efficacy of avalglucosidase alfa versus alglucosidase alfa in patients with late-onset Pompe disease (COMET): a phase 3, randomised, multicentre trial. Lancet Neurol. 2021; 20(12):1012-26.
19. Schoser B, Roberts M, Byrne B J, Sitaraman S, Jiang H, Laforet P, et al. Safety and efficacy of cipaglucosidase alfa plus miglustat versus alglucosidase alfa plus placebo in late-onset Pompe disease (PROPEL): an international, randomised, double-blind, parallel-group, phase 3 trial. Lancet Neurol. 2021; 20(12):1027-37.
20. Puertollano R, and Raben N. New therapies for Pompe disease: are we closer to a cure? Lancet Neurol. 2021; 20(12):973-5.
21. Unnisa Z, Yoon J K, Schindler J W, Mason C, and van Til N P. Gene Therapy Developments for Pompe Disease. Biomedicines. 2022; 10(2).
22. Roger A L, Sethi R, Huston M L, Scarrow E, Bao-Dai J, Lai E, et al. What's new and what's next for gene therapy in Pompe disease? Expert Opin Biol Ther. 2022; 22(9):1117-35.
23. Salabarria S M, Nair J, Clement N, Smith B K, Raben N, Fuller D D, et al. Advancements in AAV-mediated Gene Therapy for Pompe Disease. J Neuromuscul Dis. 2020; 7(1):15-31.
24. Kishnani P S, and Koeberl D D. Liver depot gene therapy for Pompe disease. Ann Transl Med. 2019; 7(13):288.
25. Colella P, and Mingozzi F. Gene Therapy for Pompe Disease: The Time is now. Hum Gene Ther. 2019; 30(10):1245-62.
26. Mingozzi F, and High K A. Overcoming the Host Immune Response to Adeno-Associated Virus Gene Delivery Vectors: The Race Between Clearance, Tolerance, Neutralization, and Escape. Annu Rev Virol. 2017; 4(1):511-34.
27. Mendell J R, Al-Zaidy S A, Rodino-Klapac L R, Goodspeed K, Gray S J, Kay C N, et al. Current Clinical Applications of In Vivo Gene Therapy with AAVs. Mol Ther. 2021; 29(2):464-88.
28. Smith B K, Collins S W, Conlon T J, Mah C S, Lawson L A, Martin A D, et al. Phase I/II trial of adeno-associated virus-mediated alpha-glucosidase gene therapy to the diaphragm for chronic respiratory failure in Pompe disease: initial safety and ventilatory outcomes. Hum Gene Ther. 2013; 24(6):630-40.
29. Corti M, Liberati C, Smith B K, Lawson L A, Tuna I S, Conlon T J, et al. Safety of Intradiaphragmatic Delivery of Adeno-Associated Virus-Mediated Alpha-Glucosidase (rAAV1-CMV-hGAA) Gene Therapy in Children Affected by Pompe Disease. Hum Gene Ther Clin Dev. 2017; 28(4):208-18.
30. LeBowitz J H, Grubb J H, Maga J A, Schmiel D H, Vogler C, and Sly W S. Glycosylation-independent targeting enhances enzyme delivery to lysosomes and decreases storage in mucopolysaccharidosis type VII mice. Proc Natl Acad Sci USA. 2004; 101(9):3083-8.
31. Raben N, Nagaraju K, Lee E, Kessler P, Byrne B, Lee L, et al. Targeted disruption of the acid alpha-glucosidase gene in mice causes an illness with critical features of both infantile and adult human glycogen storage disease type II. JBiolChem. 1998; 273(30):19086-92.
32. Davidoff A M, Ng C Y, Zhou J, Spence Y, and Nathwani A C. Sex significantly influences transduction of murine liver by recombinant adeno-associated viral vectors through an androgen-dependent pathway. Blood. 2003; 102(2):480-8.
33. Dane A P, Wowro S J, Cunningham S C, and Alexander I E. Comparison of gene transfer to the murine liver following intraperitoneal and intraportal delivery of hepatotropic AAV pseudo-serotypes. Gene Ther. 2013; 20(4):460-4.
34. Raben N, Danon M, Gilbert A L, Dwivedi S, Collins B, Thurberg B L, et al. Enzyme replacement therapy in the mouse model of Pompe disease. Mol Genet Metab. 2003; 80(1-2):159-69.
35. Meena N K, and Raben N. Pompe Disease: New Developments in an Old Lysosomal Storage Disorder. Biomolecules. 2020; 10(9).
36. Myerowitz R, Puertollano R, and Raben N. Impaired autophagy: The collateral damage of lysosomal storage disorders. EBioMedicine. 2021; 63:103166.
37. Kabeya Y, Mizushima N, Ueno T, Yamamoto A, Kirisako T, Noda T, et al. LC3, a mammalian homologue of yeast Apg8p, is localized in autophagosome membranes after processing. EMBO J. 2000; 19(21):5720-8.
38. Pankiv S, Clausen T H, Lamark T, Brech A, Bruun J A, Outzen H, et al. p62/SQSTM1 binds directly to Atg8/LC3 to facilitate degradation of ubiquitinated protein aggregates by autophagy. JBiolChem. 2007; 282(33):24131-45.
39. Bjorkoy G, Lamark T, Pankiv S, Overvatn A, Brech A, and Johansen T. Monitoring autophagic degradation of p62/SQSTM1. Methods Enzymol. 2009; 452:181-97.
40. Papadopoulos C, and Meyer H. Detection and Clearance of Damaged Lysosomes by the Endo-Lysosomal Damage Response and Lysophagy. Curr Biol. 2017; 27(24):R1330-R41.
41. Papadopoulos C, Kravic B, and Meyer H. Repair or Lysophagy: Dealing with Damaged Lysosomes. J Mol Biol. 2020; 432(1):231-9.
42. Jia J, Claude-Taupin A, Gu Y, Choi S W, Peters R, Bissa B, et al. Galectin-3 Coordinates a Cellular System for Lysosomal Repair and Removal. Dev Cell. 2020; 52(1):69-87 e8.
43. Meena N K, Ralston E, Raben N, and Puertollano R. Enzyme Replacement Therapy Can Reverse Pathogenic Cascade in Pompe Disease. Mol Ther Methods Clin Dev. 2020; 18:199-214.
44. Lim J A, Li L, Shirihai O S, Trudeau K M, Puertollano R, and Raben N. Modulation of mTOR signaling as a strategy for the treatment of Pompe disease. EMBO Mol Med. 2017.
45. Lim J A, Sun B, Puertollano R, and Raben N. Therapeutic Benefit of Autophagy Modulation in Pompe Disease. Mol Ther. 2018; 26(7):1783-96.
46. Peter D, Igreja C, Weber R, Wohlbold L, Weiler C, Ebertsch L, et al. Molecular architecture of 4E-B P translational inhibitors bound to eIF4E. Mol Cell. 2015; 57(6):1074-87.
47. Sekiyama N, Arthanari H, Papadopoulos E, Rodriguez-Mias R A, Wagner G, and Leger-Abraham M. Molecular mechanism of the dual activity of 4EGI-1: Dissociating eIF4G from eIF4E but stabilizing the binding of unphosphorylated 4E-BP1. Proc Natl Acad Sci USA. 2015; 112(30):E4036-45.
48. Raben N, Schreiner C, Baum R, Takikita S, Xu S, Xie T, et al. Suppression of autophagy permits successful enzyme replacement therapy in a lysosomal storage disorder-murine Pompe disease. Autophagy. 2010; 6(8):1078-89.
49. Harlaar L, Ciet P, van Tulder G, Brusse E, Timmermans R G M, Janssen W G M, et al. Diaphragmatic dysfunction in neuromuscular disease, an MRI study. Neuromuscul Disord. 2022; 32(1):15-24.
50. Sidman R L, Taksir T, Fidler J, Zhao M, Dodge J C, Passini M A, et al. Temporal neuropathologic and behavioral phenotype of 6neo/6neo Pompe disease mice. JNeuropatholExpNeurol. 2008; 67(8):803-18.
51. DeRuisseau L R, Fuller D D, Qiu K, DeRuisseau K C, Donnelly W H, Jr., Mah C, et al. Neural deficits contribute to respiratory insufficiency in Pompe disease. Proc Natl Acad Sci USA. 2009; 106(23):9419-24.
52. Turner S M, Hoyt A K, ElMallah M K, Falk D J, Byrne B J, and Fuller D D. Neuropathology in respiratory-related motoneurons in young Pompe (Gaa(−/−)) mice. Respir Physiol Neurobiol. 2016; 227:48-55.
53. Lee N C, Hwu W L, Muramatsu S I, Falk D J, Byrne B J, Cheng C H, et al. A Neuron-Specific Gene Therapy Relieves Motor Deficits in Pompe Disease Mice. Mol Neurobiol. 2018; 55(6):5299-309.
54. Byrne B J, Fuller D D, Smith B K, Clement N, Coleman K, Cleaver B, et al. Pompe disease gene therapy: neural manifestations require consideration of CNS directed therapy. Ann Transl Med. 2019; 7(13):290.
55. Ebbink B J, Aarsen F K, van Gelder C M, van den Hout J M, Weisglas-Kuperus N, Jaeken J, et al. Cognitive outcome of patients with classic infantile Pompe disease receiving enzyme therapy. Neurology. 2012; 78(19):1512-8.
56. Lim J A, Yi H, Gao F, Raben N, Kishnani P S, and Sun B. Intravenous Injection of an AAV-PHP.B Vector Encoding Human Acid alpha-Glucosidase Rescues Both Muscle and CNS Defects in Murine Pompe Disease. Mol Ther Methods Clin Dev. 2019; 12:233-45.
57. Wisselaar H A, Kroos M A, Hermans M M, van Beeumen J, and Reuser A J. Structural and functional changes of lysosomal acid alpha-glucosidase during intracellular transport and maturation. JBiolChem. 1993; 268(3):2223-31.
58. Eggers M, Vannoy C H, Huang J, Purushothaman P, Brassard J, Fonck C, et al. Muscle-directed gene therapy corrects Pompe disease and uncovers species-specific GAA immunogenicity. EMBO Mol Med. 2022; 14(1):e13968.
59. Korlimarla A, Lim J A, Kishnani P S, and Sun B. An emerging phenotype of central nervous system involvement in Pompe disease: from bench to bedside and beyond. Ann Transl Med. 2019; 7(13):289.
60. Franco L M, Sun B, Yang X, Bird A, Zhang H, Schneider A, et al. Evasion of immune responses to introduced human acid alpha-glucosidase by liver-restricted expression in glycogen storage disease type II. MolTher. 2005; 12(5):876-84.
61. Sun B, Chen Y T, Bird A, Amalfitano A, and Koeberl D D. Long-term correction of glycogen storage disease type II with a hybrid Ad-AAV vector. MolTher. 2003; 7(2):193-201.
62. Ziegler R J, Bercury S D, Fidler J, Zhao M A, Foley J, Taksir T V, et al. Ability of adeno-associated virus serotype 8-mediated hepatic expression of acid alpha-glucosidase to correct the biochemical and motor function deficits of presymptomatic and symptomatic Pompe mice. HumGene Ther. 2008; 19(6):609-21.
63. Sun B, Zhang H, Benjamin D K, Jr., Brown T, Bird A, Young S P, et al. Enhanced efficacy of an AAV vector encoding chimeric, highly secreted acid alpha-glucosidase in glycogen storage disease type II. MolTher. 2006; 14(6):822-30.
64. Puzzo F, Colella P, Biferi M G, Bali D, Paulk N K, Vidal P, et al. Rescue of Pompe disease in mice by AAV-mediated liver delivery of secretable acid alpha-glucosidase. Sci Transl Med. 2017; 9(418).
65. Puertollano R, and Raben N. Pompe disease: how to solve many problems with one solution. Ann Transl Med. 2018; 6(15):313.
66. Ruzo A, Garcia M, Ribera A, Villacampa P, Haurigot V, Marco S, et al. Liver production of sulfamidase reverses peripheral and ameliorates CNS pathology in mucopolysaccharidosis IIIA mice. Mol Ther. 2012; 20(2):254-66.
67. Cagin U, Puzzo F, Gomez M J, Moya-Nilges M, Sellier P, Abad C, et al. Rescue of Advanced Pompe Disease in Mice with Hepatic Expression of Secretable Acid alpha-Glucosidase. Molecular Therapy. 2020; 28(9):2056-72.
68. Costa-Verdera H, Collaud F, Riling C R, Sellier P, Nordin J M L, Preston G M, et al. Hepatic expression of GAA results in enhanced enzyme bioavailability in mice and non-human primates. Nat Commun. 2021; 12(1):6393.
69. Duan D. Systemic delivery of adeno-associated viral vectors. Curr Opin Virol. 2016; 21:16-25.
70. Raben N, Jatkar T, Lee A, Lu N, Dwivedi S, Nagaraju K, et al. Glycogen stored in skeletal but not in cardiac muscle in acid alpha-glucosidase mutant (Pompe) mice is highly resistant to transgene-encoded human enzyme. MolTher. 2002; 6(5):601-8.
71. Devi G R, Byrd J C, Slentz D H, and MacDonald R G. An insulin-like growth factor II (IGF-II) affinity-enhancing domain localized within extracytoplasmic repeat 13 of the IGF-II/mannose 6-phosphate receptor. Mol Endocrinol. 1998; 12(11):1661-72.
72. Bohnsack R N, Song X, Olson L J, Kudo M, Gotschall R R, Canfield W M, et al. Cation-independent mannose 6-phosphate receptor: a composite of distinct phosphomannosyl binding sites. J Biol Chem. 2009; 284(50):35215-26.
73. Yogalingam G, Luu A R, Prill H, Lo M J, Yip B, Holtzinger J, et al. BMN 250, a fusion of lysosomal alpha-N-acetylglucosaminidase with IGF2, exhibits different patterns of cellular uptake into critical cell types of Sanfilippo syndrome B disease pathogenesis. PLoS One. 2019; 14(1):e0207836.
74. Doyle B M, Turner S M F, Sunshine M D, Doerfler P A, Poirier A E, Vaught L A, et al. AAV Gene Therapy Utilizing Glycosylation-Independent Lysosomal Targeting Tagged GAA in the Hypoglossal Motor System of Pompe Mice. Mol Ther Methods Clin Dev. 2019; 15:194-203.
75. Lee K Z, Qiu K, Sandhu M S, Elmallah M K, Falk D J, Lane M A, et al. Hypoglossal neuropathology and respiratory activity in pompe mice. Front Physiol. 2011; 2:31.
76. van Gelder C M, van Capelle C I, Ebbink B J, Moor-van Nugteren I, van den Hout J M, Hakkesteegt M M, et al. Facial-muscle weakness, speech disorders and dysphagia are common in patients with classic infantile Pompe disease treated with enzyme therapy. J Inherit Metab Dis. 2012; 35(3):505-11.
77. Chan J, Desai A K, Kazi Z B, Corey K, Austin S, Hobson-Webb L D, et al. The emerging phenotype of late-onset Pompe disease: A systematic literature review. Mol Genet Metab. 2017; 120(3):163-72.
78. Dogan Y, Barese C. N., Schindler, J. W., Toon, J. K., Unnisa, Z., Guda, S., Jacobs, M. E., Oborski, C., Clarke, D. L., Schambach, A., Pfeifer, R., Harper, C., Mason, C., vanTil N. P. Screening of Chimeric GAA Variants in a Preclinical Study of Pompe Disease Results in Candidate Vector for Hematopoietic Stem Cell Gene Therapy bioRxiv. 2021.
79. Stok M, de Boer H, Huston M W, Jacobs E H, Roovers O, Visser T P, et al. Lentiviral Hematopoietic Stem Cell Gene Therapy Corrects Murine Pompe Disease. Mol Ther Methods Clin Dev. 2020; 17:1014-25.
80. Piras G, Montiel-Equihua C, Chan Y A, Wantuch S, Stuckey D, Burke D, et al. Lentiviral Hematopoietic Stem Cell Gene Therapy Rescues Clinical Phenotypes in a Murine Model of Pompe Disease. Mol Ther Methods Clin Dev. 2020; 18:558-70.
81. Liang Q, Vlaar E C, Catalano F, Pijnenburg J M, Stok M, van Helsdingen Y, et al. Lentiviral gene therapy prevents anti-human acid alpha-glucosidase antibody formation in murine Pompe disease. Mol Ther Methods Clin Dev. 2022; 25:520-32.
82. Peng J, Dalton J, Butt M, Tracy K, Kennedy D, Haroldsen P, et al. Reveglucosidase alfa (BMN 701), an IGF2-Tagged rhAcid alpha-Glucosidase, Improves Respiratory Functional Parameters in a Murine Model of Pompe Disease. J Pharmacol Exp Ther. 2017; 360(2):313-23.
83. Byrne B J, Geberhiwot T, Barshop B A, Barohn R, Hughes D, Bratkovic D, et al. A study on the safety and efficacy of reveglucosidase alfa in patients with late-onset Pompe disease. Orphanet J Rare Dis. 2017; 12(1):144.
84. Schoser B, Hill V, and Raben N. Therapeutic approaches in glycogen storage disease type II/Pompe Disease. Neurotherapeutics. 2008; 5(4):569-78.
85. Koeberl D D, Luo X, Sun B, McVie-Wylie A, Dai J, Li S, et al. Enhanced efficacy of enzyme replacement therapy in Pompe disease through mannose-6-phosphate receptor expression in skeletal muscle. Molecular genetics and metabolism. 2011; 103(2):107-12.
86. Mendell J R, Al-Zaidy S, Shell R, Arnold W D, Rodino-Klapac L R, Prior T W, et al. Single-Dose Gene-Replacement Therapy for Spinal Muscular Atrophy. N Engl J Med. 2017; 377(18):1713-22.
87. Day J W, Finkel R S, Chiriboga C A, Connolly A M, Crawford T O, Darras B T, et al. Onasemnogene abeparvovec gene therapy for symptomatic infantile-onset spinal muscular atrophy in patients with two copies of SMN2 (STRIVE): an open-label, single-arm, multicentre, phase 3 trial. Lancet Neurol. 2021; 20(4):284-93.
88. Amalfitano A, McVie-Wylie A J, Hu H, Dawson T L, Raben N, Plotz P, et al. Systemic correction of the muscle disorder glycogen storage disease type II after hepatic targeting of a modified adenovirus vector encoding human acid-alpha-glucosidase. ProcNatlAcadSciUSA. 1999; 96(16):8861-6.
89. Kikuchi T, Yang H W, Pennybacker M, Ichihara N, Mizutani M, Van Hove J L, et al. Clinical and metabolic correction of pompe disease by enzyme therapy in acid maltase-deficient quail. JClinInvest. 1998; 101(4):827-33.
90. Raben N, Shea L, Hill V, and Plotz P. Monitoring autophagy in lysosomal storage disorders. Methods Enzymol. 2009; 453:417-49.
91. Feeney E J, Austin S, Chien Y H, Mandel H, Schoser B, Prater S, et al. The value of muscle biopsies in Pompe disease: identifying lipofuscin inclusions in juvenile- and adult-onset patients. Acta Neuropathol Commun. 2014; 2(1):2-17.

We claim:

1. A gene therapy vector comprising a nucleic acid construct encoding a polypeptide comprising:
a therapeutic protein comprising acid alpha-glucosidase (GAA);
a CIMPR-binding peptide comprising a variant IGF2 (vIGF2) peptide; and
a signal peptide comprising a binding immunoglobulin protein (BiP) signal peptide.

2. The gene therapy vector of claim 1, further comprising an adeno-associated viral (AAV) vector.

3. The gene therapy vector of claim 2, wherein the AAV vector is an AAV9 vector.

4. The gene therapy vector of any one of claims 1-3, further comprising a promoter.

5. The gene therapy vector of claim 4, wherein the promoter comprises a cytomegalovirus (CMV) early enhancer/chicken R actin (CAG) promoter.

6. The gene therapy vector of any one of claims 1-5, wherein the GAA has an amino acid sequence at least 90% identical to a sequence selected from the group consisting of SEQ ID NOS: 1-4.

7. The gene therapy vector of any one of claims 1-6, wherein the GAA has an amino acid sequence at least 95% identical to a sequence selected from the group consisting of SEQ ID NOS: 1-4.

8. The gene therapy vector of any one of claims 1-7, wherein the GAA has an amino acid sequence selected from the group consisting of SEQ ID NOS: 1-4.

9. The gene therapy vector of any one of claims 1-8, wherein the vIGF2 peptide has an amino acid sequence at least 90% identical to a sequence selected from the group consisting of SEQ ID NOS: 5-19.

10. The gene therapy vector of any one of claims 1-9, wherein the vIGF2 peptide has an amino acid sequence at least 95% identical to a sequence selected from the group consisting of SEQ ID NOS: 5-19.

11. The gene therapy vector of any one of claims 1-10, wherein the vIGF2 peptide has an amino acid sequence selected from the group consisting of SEQ ID NOS: 5-19.

12. The gene therapy vector of any one of claims 1-11, wherein the nucleic acid encoding the vIGF2 peptide has a nucleic acid sequence at least 90% identical to a sequence selected from the group consisting of SEQ ID NOS 25 or 26.

13. The gene therapy vector of any one of claims 1-8, wherein the BiP signal peptide has an amino acid sequence at least 90% identical to a sequence selected from the group consisting of SEQ ID NOS: 20-24.

14. The gene therapy vector of any one of claims 1-9, wherein the BiP signal peptide has an amino acid sequence at least 95% identical to a sequence selected from the group consisting of SEQ ID NOS: 20-24.

15. The gene therapy vector of any one of claims 1-10, wherein the BiP signal peptide has an amino acid sequence selected from the group consisting of SEQ ID NOS: 20-24.

16. A method of treating Pompe disease in a subject, the method comprising administering a therapeutically effective amount of the gene therapy vector of any one of claims 1-15.

17. The method of claim 16, wherein the gene therapy vector is administered systemically.

18. The method of claim 16 or 17, wherein the therapeutically effective amount of the gene therapy vector is in a range of from about 1×10¹² to about 1×10¹⁵ vg.

19. The method of any one of claims 6-8, wherein the therapeutically effective amount of the gene therapy vector is in a range of from about 5×10¹² to about 1×10¹⁴ vg.

20. The method of any one of claims 16-19, wherein the therapeutically effective amount of the composition is about 2.5×10¹³ vg or about 5×10¹³ vg.

21. The method of any one of claims 16-20, wherein the gene therapy vector is administered via a route selected from the group consisting of intrathecal, intracisternal, lumbar puncture, intracranial, intracerebroventricular, intraparenchymal, intravenous and combinations thereof.

22. The method of any one of claims 16-21, wherein gene therapy vector is not administered in combination with a non-ionic, low-osmolar contrast agent.

23. The method of any one of claims 16-21, wherein gene therapy vector is administered in combination with a non-ionic, low-osmolar contrast agent.

24. The method of claim 23, wherein the non-ionic, low-osmolar contrast agent is selected from the group consisting of iobitridol, iohexol, iomeprol, iopamidol, iopentol, iopromide, ioversol, ioxilan, and combinations thereof.

25. The method of any one of claims 16-24, wherein the method reduces glycogen storage in patient cells.

26. The method of any one of claims 16-25, wherein the method reduces glycogen storage in one or more of skeletal and cardiac muscles, the diaphragm, and the central nervous system (CNS).


SEQUENCE LISTING (26 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - protein, 896 aa, Homo sapiens
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 4 - protein, 896 aa, Homo sapiens
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 5 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 6 - protein, 67 aa, Homo sapiens
AYRPSETLCGGELVDTLQFVCGDRGSYFSRPASRVSRRSRGIVEECCFRSCDLALLETYCATPAKSE

SEQ ID NO: 7 - protein, 67 aa, Homo sapiens
AYRPSETLCGGELVDTLQFVCGDRGFLFSRPASRVSRRSRGIVEECCFRSCDLALLETYCATPAKSE

SEQ ID NO: 8 - protein, 67 aa, Homo sapiens
AYRPSETLCGGELVDTLQFVCGDRGFYFSRPASRVSRRSRGILEECCFRSCDLALLETYCATPAKSE

SEQ ID NO: 9 - protein, 67 aa, Homo sapiens
AYRPSETLCGGELVDTLQFVCGDRGFYFSRPASRVSRRSRGIVEECCTRSCDLALLETYCATPAKSE

SEQ ID NO: 10 - protein, 67 aa, Homo sapiens
AYRPSETLCGGELVDTLQFVCGDRGFYFSRPASRVSRRSRGIVEECCFSSCDLALLETYCATPAKSE

SEQ ID NO: 11 - protein, 67 aa, Homo sapiens
AYRPSETLCGGELVDTLQFVCGDRGFYFSRPASRVSRRSRGIVEECCFRICDLALLETYCATPAKSE

SEQ ID NO: 12 - protein, 67 aa, Homo sapiens
AYRPSETLCGGELVDTLQFVCGDRGFYFSRPASRVSRRSRGIVEECCFRSCDLRLLETYCATPAKSE

SEQ ID NO: 13 - protein, 66 aa, Homo sapiens
AYRPSETLCGGELVDTLQFVCGDRGFYFSRPASRVSRRSRGIVEECCFRSCDLARLETYCATPAKS

SEQ ID NO: 14 - protein, 67 aa, Homo sapiens
AYRPSETLCGGELVDTLQFVCGDRGSLFSRPASRVSRRSRGILEECCTSICDLRRLETYCATPAKSE

SEQ ID NO: 15 - protein, 61 aa, Homo sapiens
TLCGGELVDTLQFVCGDRGFLFSRPASRVSRRSRGIVEECCFRSCDLALLETYCATPARSE

SEQ ID NO: 16 - protein, 60 aa, Homo sapiens
LCGGELVDTLQFVCGDRGFLFSRPASRVSRRSRGIVEECCFRSCDLALLETYCATPARSE

SEQ ID NO: 17 - protein, 63 aa, Homo sapiens
SRTLCGGELVDTLQFVCGDRGFLFSRPASRVSRRSRGIVEECCFRSCDLALLETYCATPARSE

SEQ ID NO: 18 - protein, 63 aa, Homo sapiens
SRTLCGGELVDTLQFVCGDRGFLFSRPASRVSRRSRGIVEECCFRSCDLALLETYCATPAKSE

SEQ ID NO: 19 - protein, 67 aa, Homo sapiens
AYRPSRTLCGGELVDTLQFVCGDRGFYFSRPASRVSRRSRGIVEECCFRSCDLALLETYCATPAKSE

SEQ ID NO: 20 - protein, 18 aa, Homo sapiens
MKLSLVAAMLLLLSAARA

SEQ ID NO: 21 - protein, 28 aa, Homo sapiens
MKLSLVAAMLLLLSLVAAMLLLLSAARA

SEQ ID NO: 22 - protein, 25 aa, Homo sapiens
MKLSLVAAMLLLLWVALLLLSAARA

SEQ ID NO: 23 - protein, 26 aa, Homo sapiens
MKLSLVAAMLLLLSLVALLLLSAARA

SEQ ID NO: 24 - protein, 26 aa, Homo sapiens
MKLSLVAAMLLLLALVALLLLSAARA

SEQ ID NO: 25 - DNA, 201 bp, Homo sapiens
  source 1..201: mol_type = unassigned DNA
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 26 - DNA, 189 bp, Homo sapiens
  source 1..189: mol_type = unassigned DNA
tctagaacactgtgcggaggggagcttgtagacactcttcagttcgtgtgtggagatcgcgggttcctcttctctcgccccgcttccagagtttcacggaggtctaggggtatagtagaggagtgttgtttcaggtcctgtgacttggcgctcctcgagacctattgcgcgacgccagccaggtccgaa